(19) United States
(12) Patent Application Publication
(10) Pub. No.: US 2012/0171172 A1
(43) Pub. Date: Jul. 5, 2012

(54) Methods Of Engineering Neural Tissue

(76) Inventors: Maria Siemionow, Shaker Heights, OH (US); Siemionow Krzysztof, Shaker Heights, OH (US)

(21) Appl. No.: 13/393,750

(22) PCT Filed: Sep. 1, 2010
(86) PCT No.: PCT/US10/47547

§ 371 Date: Mar. 1, 2012

Related U.S. Application Data
(60) Provisional application No. 61/239,977, filed on Sep. 4, 2009.

(51) Int. Cl. A61K 35/30; C12N 5/079

(52) U.S. Cl. 424/93.7; 435/325

(57) ABSTRACT

In one aspect, the invention is a method of generating a neural conduit comprising neurotrophic factors and angiogenic factors ex vivo comprising introducing cells that enhance nerve regeneration into an isolated, naturally occurring epineural sheath, thereby producing a combination. The combination is maintained under conditions in which neurotrophic factors and angiogenic factors are expressed in the epineural sheath, thereby generating a neural conduit comprising neurotrophic factors and angiogenic factors ex vivo. In another aspect, the invention is directed to neural conduits such as a neural conduit produced by the methods provided herein. In other aspects, the invention is directed to an article of manufacture.

### RELATED APPLICATIONS

This application claims the benefit of U.S. Provisional Application No. 61/239,977, filed on Sep. 4, 2009. The entire teachings of the above application is incorporated herein by reference.

### BACKGROUND OF THE INVENTION

Due to the lack of available and effective neural conduits for the generation of neural tissue, there has been increasing interest in creating synthetic tubes that can mimic the natural biological milieu. These tubes can be created from a wide variety of substrates and include different bio-engineered properties. These properties generally attempt to augment neural regeneration through the use of neurotrophic factors (NFs), cells, or genes. These methods can further be divided by whether the material is introduced or fixed into the conduit and by the timing of delivery. The material needed to make an effective conduit has to provide structural integrity while maintaining pliability. In addition, the material should also be amenable to contact guidance and especially over long defects, semi-permeable. The conduit's permeability should maximize the neurotrophic concentrations and minimize harmful, inflammatory, molecule infiltration. Synthetic conduits should also have very low immunogenicity to decrease the risk of a harmful host response which could potentially necessitate removal. One of the first attempts at using a bio-synthetic conduit was the use of silicone. In general, silicone is malleable and relatively well tolerated by the host. Studies have demonstrated that using a silicone conduit resulted in similar functional recoveries after repairing small gaps compared to traditional microsurgical techniques. However, the silicone conduits have not been proven useful in more extensive gaps, possibly due to its low permeability and lack of inherent neurotrophism. Silicone conduits have also been associated with chronic nerve compression, irritation requiring removal, inflammation, and fibrosis. In two clinical studies, 25-50% of silicone implants had to be explanted due to patient discomfort. This has led some investigators to use bio-degradable tubes that can provide the necessary scaffold and micro-environment for regeneration, but can be broken down by the body's natural enzymes. A list of sufficiently studied materials includes: poly(3-hydroxybutyrate), Chitosan, D-glucosamine, polyester urethane, poly(2-hydroxymethyl methacrylate-co-methyl mehacrylate), poly-lactic (PLA), poly-glycolic (PGA), and polylactic-co-glycolic) acid (PLGA) tubes (e.g. Maxon). While study results have varied with different regeneration profiles, these conduits have been largely unsuccessful in longer defects. In response to these shortcomings, other more natural materials have been investigated. Collagen, fibronectin, and laminin conduits have been used with demonstrable regeneration. The improved biocompatibility is thought to actively promote the migration of Schwann cells and enhance contact guidance for axonal growth. Bovine collagen has been marketed under the name NeuroGen®, but has yet to undergo clinical trials and carries the potential risk of harboring prion disease. However, even with the increased bio-compatibility of these substances, success in repair of nerve gaps over 3 cm has yet to be achieved.

Continued research in this area has led to developing not only combinations of bio-synthetic and naturally occurring bio-compatible materials but also integrated delivery systems for neurotrophic factors and neurotrophic-producing cells. However, replicating the optimal concentrations, kinetics, and gradients has proven to be extremely difficult. Both osmotic pumps and injection devices have been studied in vitro, with each having its own set of limitations. Osmotic pumps are limited by zero-order kinetics and injection devices are relegated to providing only burst release. Therefore, the ability to deliver the appropriate doses, often in the nano- to micro-molar concentrations, with the appropriate time sequence and interval is not yet possible using these devices. In addition, both have the added disadvantages of size, need for catheter placement and immobilization while implanted. In recent years, the use of micro-spheres has been studied in attempts to provide more regulated and longer acting delivery of neurotrophic factors.

While these novel modes of delivery are very exciting, they have yet to be perfected and have the drawbacks of difficult manufacturing and sterilization.

### SUMMARY OF THE INVENTION

Provided herein are compositions comprising engineered neural tissue and methods for engineering neural tissue. In one aspect, the invention is directed to a method of generating a neural conduit comprising neurotrophic factors and angiogenic factors ex vivo comprising maintaining an isolated, naturally occurring epineural sheath (e.g., an isolated, naturally occurring epineural tube; an isolated, naturally occurring epineural patch) under conditions in which neurotrophic factors and/or angiogenic factors are expressed in the epineural sheath, thereby generating a neural conduit comprising neurotrophic factors and/or angiogenic factors ex vivo. Conditions in which neurotrophic factors and/or angiogenic factors are expressed in the epineural sheath comprise contacting the epineural sheath with saline, epineurium (e.g., epineurol strip(s); epineural powder) or a combination thereof, culturing the tube for about 3 days, 7 days, 14 days, 21 days, 28 days or 35 days in a cell culture medium at a temperature of about 37° C. in 5% CO₂ or combinations thereof.

In a particular aspect, the invention is directed to a method of generating a neural conduit comprising neurotrophic factors and angiogenic factors ex vivo comprising expanding the volume of an isolated, naturally occurring epineural tube (e.g., introducing saline, strips of epineurium, and/or epinueral powder) thereby producing an expanded epineural tube, and maintaining the expanded epineural tube under conditions in which neurotrophic factors and/or angiogenic factors are expressed in the epineural tube, thereby generating a neural conduit comprising neurotrophic factors and/or angiogenic factors ex vivo.

In another aspect, cells that enhance nerve generation or regeneration (e.g., naïve BMSCs) can be induced into expressing neurotrophic and angiogenic factors ex vivo solely by exposure to the inherent neurogenic properties of an epineural sheath. Cells such as naïve BMSCs are bio-responsive and display different phenotypic properties based on exposure and can both grow and expand in the micro-environment provided by the epineural sheath. As shown herein, the combination of cells such as BMSCs and an epineural sheath ex vivo can be used to bridge nerve defects in vivo without negative sequelae. Combining cells that enhance nerve generation or regeneration (e.g., BMSCs) with an epineural sheath ex vivo will lead to enhanced functional recovery (sensory and motor) and neural regeneration in vivo.

Accordingly, one aspect of the invention is a method of generating a neural conduit comprising neurotrophic factors and angiogenic factors ex vivo comprising introducing cells that enhance nerve regeneration into an isolated, naturally occurring epineural sheath, thereby producing a combination. The combination is maintained under conditions in which neurotrophic factors and angiogenic factors are expressed in the epineural sheath, thereby generating a neural conduit comprising neurotrophic factors and angiogenic factors ex vivo.

In another aspect, the invention is directed to a neural conduit produced by the methods provided herein.

In another aspect, the invention is directed to a neural conduit comprising an isolated naturally occurring epineural sheath and cells which enhance neural regeneration.

In other aspects, the invention is directed to an article of manufacture comprising one or more isolated naturally occurring epineural sheaths, a device for introducing an (one or more) agent (e.g., cells) which enhance neural regeneration into the one or more epineural sheaths, and instructions for use thereof. In another aspect, the article of manufacture comprises one or more neural conduits, wherein each neural conduit comprises an isolated naturally occurring epineural tube and cells which enhance neural regeneration, and instructions for use thereof. The article of manufacture can further comprise a device for introducing an agent which enhances neural regeneration into the one or more epineural sheaths. In yet another aspect, the article of manufacture comprises a neural conduit which contains neurotrophic factors, angiogenic factors or a combination thereof, and instructions for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Bone Marrow Stromal Cell Preparation (BMSC): Rat femur and tibia bones were aseptically flushed using alpha-MEM medium followed by red blood cell lysis and transferred to adherent flasks containing alpha-MEM complete medium which was exchanged 3 times/week.

FIGS. 2A-2C shows epineural sheath harvesting technique: (2A) The dissected rat sciatic nerve was suspended on a straight irrigator. (2B) While suspended, nerve fibers were extirpated using forceps leaving (2C) an empty epineural tube.

FIG. 3 shows Stromal Cell Epineural Conduit (SCEC) creation: BMSCs were stained with PKH-26 red dye and transferred to a syringe. An empty epineural sheath was suspended on a needle and the distal end was ligated followed by insufflation with Lactated Ringers. BMSCs were then injected into the epineural sheath while ligating the proximal end and then transferred to a non-adherent flask filled with medium that was exchanged every 2-3 days for 2 weeks. After the epineural sheath was harvested, and all the fasiscles were removed, the distal end was suture ligated. A 25 gauge syringe was filled with 0.2 ml of saline injected into the sheath (or following saline injection to expand the tube, epineural strip/strips were inserted or epineural powder was injected) and the proximal end was then ligated. The tube was than transferred to an non-adherent flask filled with medium that is exchanged every 2-3 days for 2 weeks.

FIGS. 4A-4C shows Stromal Cell Epineural Conduit (SCEC) preparation for transplantation: (4A) The SCEC was gently removed from the flask after 14 day co-culture. (4B) The length of the SCEC was measured and a comparable defect was created in the right sciatic nerve of a naïve Lewis recipient. (4C) The SCEC was then coaptated into the resulting defect using 10-0 suture.

FIG. 5 shows sensory recovery testing using Pin-Prick (PP): Forceps were applied to the foot of the rat and observed for limb retraction. PP was measured from proximal to distal and assigned values: 0=no response, 1=heel, 2=mid-foot, and 3=toes.

FIG. 6 shows motor recovery testing using Toe-Spread (TS): The rat was gently suspended rostrally and the assigned a TS score: 0=no extension, 1=any movement, 2=toe abduction, and 3=toe abduction with limb extension.

FIG. 7 shows Somato-Sensory Evoked Potential (SSEP) recording: stimulation of the sciatic nerve in the hind limb with measurement electrodes at the level of the somatosensory cortex.

FIG. 8 shows immunohistochemical evaluation of ex vivo allogenic (ACI) sheath co-cultured with isogenic (Lew) BMSCs after 3, 7, and 14 days: The presence of neurotrophic/angiogenic factors (green) were assessed using a fluorescent microscope as well as the presence of BMSCs (red) and BMSC/factor co-expression (orange).

FIG. 9 shows ex vivo cultured Stromal Cell Epineural Conduit (SCEC): Allogenic tube (ACI)/Isogenic BMSCs (Lew) after 14 days culture stained with Toluidine Blue (100× magnification).

FIG. 10 shows regenerative potential of in vivo transplanted Stromal Cell Epineural Conduit (SCEC): Allogenic tube (ACI)/isogenic BMSCs (Lew) after 14 days culture ex vivo was transplanted to a naïve Lewis recipient following sciatic nerve transection. SCEC was harvested 6 weeks post-transplant and stained with Toluidine Blue (100× magnification).

FIG. 11 shows Myelin, Axon, and Nerve Areas: Comparison between transplanted allogenic tube (ACI) filled with isogenic stromal cells after 14 days culture (Stromal Cell Epineural Conduit—SCEC), allogenic (ACI) epineurium with immediate injection of isogenic (Lew) stromal cells (Allo-tube Iso), and allogenic (ACI) epineurium with immediate injection of saline (Allo-tube Sal) harvested at 6 weeks. Proximal (P), Middle (M), and Distal (D) sections of transplanted epineurium were stained with Toluidine Blue and viewed under a light microscope. Six images were taken per sample and analyzed using Image Pro-Plus.

FIG. 12 shows Myelin Thickness: Comparison between transplanted allogenic tube (ACI) filled with isogenic stromal cells after 14 days culture (Stromal Cell Epineural Conduit—SCEC), allogenic (ACI) epineurium with immediate injection of isogenic (Lew) stromal cells (Allo-tube Iso), and allogenic (ACI) epineurium with immediate injection of saline (Allo-tube Sal) harvested at 6 weeks. Proximal (P), Middle (M), and Distal (D) sections of transplanted epineurium were stained with Toluidine Blue and viewed under a light microscope. Six images were taken per sample and analyzed using Image Pro-Plus.

FIG. 13 shows Mean Fiber and Axon diameters: Comparison between transplanted allogenic tube (ACI) filled with isogenic stromal cells after 14 days culture (Stromal Cell Epineural Conduit—SCEC), allogenic (ACI) epineurium with immediate injection of isogenic (Lew) stromal cells (Allo-tube Iso), and allogenic (ACI) epineurium with immediate injection of saline (Allo-tube Sal) harvested at 6 weeks. Proximal (P), Middle (M), and Distal (D) sections of transplanted epineurium were stained with Toluidine Blue and viewed under a light microscope. Six images were taken per sample and analyzed using Image Pro-Plus.

FIG. 14 shows Number of axons: Comparison between transplanted allogenic tube (ACI) filled with isogenic stromal cells after 14 days culture (Stromal Cell Epineural Conduit—SCEC), allogenic (ACI) epineurium with immediate injection of isogenic (Lew) stromal cells (Allo-tube Iso), and allogenic (ACI) epineurium with immediate injection of saline (Allo-tube Sal) harvested at 6 weeks. Proximal (P), Middle (M), and Distal (D) sections of transplanted epineurium were stained with Toluidine Blue and viewed under a light microscope. Six images were taken per sample and analyzed using Image Pro-Plus.

FIG. 15 shows Axonal Density: Comparison between transplanted allogenic tube (ACI) filled with isogenic stromal cells after 14 days culture (Stromal Cell Epineural Conduit—SCEC), allogenic (ACI) epineurium with immediate injection of isogenic (Lew) stromal cells (Allo-tube Iso), and allogenic (ACI) epineurium with immediate injection of saline (Allo-tube Sal) harvested at 6 weeks. Proximal (P), Middle (M), and Distal (D) sections of transplanted epineurium were stained with Toluidine Blue and viewed under a light microscope. Six images were taken per sample and analyzed using Image Pro-Plus.

FIGS. 16A-16D show immunocytochemical analysis of CD31 in epineural tube filled with stromal cells and maintained in culture for 12 days. Epineurial tube was isolated from sciatic nerve of ACI rats and stromal cells were prepared from bone marrow of ACI rat. ACI/ACI 1 (16A and 16B) and ACI/ACI 2 (16C and 16D) represent two independently cultured ACI epineural tubes filled with ACI stromal cells. Peroxidase staining.

FIGS. 17A-17B show immunocytochemical analysis of CD31 in rat's sciatic nerve isolated from two ACI rats (17A and 17B). Peroxidase staining.

FIGS. 18A-18C show immunocytochemical analysis of Laminin 2 in ACI rat's sciatic nerve (18A), epineural tube from Lewis rat: LEW-ET (18B) and 12 days cultured ACI rat epineural tube filled with ACI stromal cells: ACI/ACI (18C). FITC immunofluorescence staining.

FIGS. 19A-19C show immunocytochemical analysis of GFAP in ACI rat's sciatic nerve (19A), epineural tube from Lewis rat: LEW-ET (19B) and 12 days cultured ACI rat epineural tube filled with ACI stromal cells: ACI/ACI (19C). FITC immunofluorescence staining.

FIGS. 20A-20C show immunocytochemical analysis of NGF in ACI rat's sciatic nerve (20A), epineural tube from Lewis rat: LEW-ET (20B) and 12 days cultured ACI rat epineural tube filled with ACI stromal cells: ACI/ACI (20C). FITC immunofluorescence staining.

FIGS. 21A-21C show immunocytochemical analysis of VEGF in ACI rat's sciatic nerve (21A), epineural tube from Lewis rat: LEW-ET (21B) and 12 days cultured ACI rat epineural tube filled with ACI stromal cells: ACI/ACI (21C). FITC immunofluorescence staining.

FIGS. 22A-22B show immunocytochemical analysis of S-100 in ACI rat's sciatic nerve (22A) and 12 days cultured ACI rat epineural tube filled with ACI stromal cells: ACl/ACI (22B). FITC immunofluorescence staining.

FIGS. 23A-23C show immunocytochemical analysis of von Willebrandt factor (WF) in ACI rat's sciatic nerve (23A), epineural tube from Lewis rat: LEW-ET (23B) and 12 days cultured ACI rat epineural tube filled with ACI stromal cells: ACI/ACI (23C). FITC immunofluorescence staining.

FIGS. 24A-24B show immunostaining data after engineered conduits were implanted into rats and evaluated 12 weeks after transplantation showing potential for nerve regeneration and expression of growth factors supporting nerve regeneration.

FIGS. 25A-25F show the ELISA results of the epineural tubes cultured ex vivo.

### DETAILED DESCRIPTION OF THE INVENTION

In response to the shortcomings of current therapies, provided herein are compositions comprising engineered neural tissue and methods for engineering neural tissue. In one aspect, the invention is directed to a method of generating a neural conduit comprising neurotrophic factors and angiogenic factors ex vivo comprising maintaining an isolated, naturally occurring epineural sheath (e.g., an isolated, naturally occurring epineural tube; an isolated, naturally occurring epineural patch) under conditions in which neurotrophic factors and/or angiogenic factors are expressed in the epineural sheath, thereby generating a neural conduit comprising neurotrophic factors and/or angiogenic factors ex vivo. Conditions in which neurotrophic factors and/or angiogenic factors are expressed in the epineural sheath comprise contacting the epineural sheath with saline, epineurium (e.g., epineurol strip(s); epineural powder) or a combination thereof, culturing the tube for about 3 days, 7 days, 14 days, 21 days, 28 days or 35 days in a cell culture medium at a temperature of about 37° C. in 5% CO₂.

In a particular aspect, the invention is directed to a method of generating a neural conduit comprising neurotrophic factors and angiogenic factors ex vivo comprising expanding the volume of an isolated, naturally occurring epineural tube thereby producing an expanded epineural tube, and maintaining the expanded epineural tube under conditions in which neurotrophic factors and/or angiogenic factors are expressed in the epineural tube, thereby generating a neural conduit comprising neurotrophic factors and/or angiogenic factors ex vivo. As will be appreciated by those of skill in the art, a variety of methods can be used to expand an epineural tube. Examples of such techniques include introducing a filler into the tube such as saline, strips of epineurium, epinueral powder.

In another aspect, a natural conduit augmented with cells that enhance nerve generation (e.g, bone marrow stromal cells) ex vivo has been created e.g., for use in treating peripheral nerve defects. The conduit is an epineural sheath that provides an ideal microenvironment for nerve regeneration as it not only protects against local fibrotic and inflammatory insults but also provides a source of growth factors crucial to effective nerve regeneration. Unlike artery and vein grafting, harvesting of the epineural sheath results in a lesser degree of donor site morbidity and has the potential to be an unlimited conduit resource via cadaveric harvesting. Multi-potent cells such as bone marrow stromal cells (BMSCs) have the ability to differentiate into several cell lineages. Cells such as BMSCs can enhance or augment neural regeneration through the production and/or stimulation of nerve growth factors (NFs), anti-inflammatory effects, and differentiation into neural support cells. Cells such as BMSCs can be reliably isolated and cultured for use in either isogenic or allogenic models.

In particular aspects, provided herein are methods of generating a neural conduit comprising neurotrophic factors, angiogenic factors or a combination thereof ex vivo by combining the neurotrophic effects of an isolated, naturally occurring epineural sheath with cells that enhance nerve generation (e.g., naïve bone marrow stromal cells). These components were combined ex vivo and shown to express neurotrophic factors and angiogenic factors ex vivo prior to implantation, and enhance neural regeneration in vivo post implantation.

“Neurotrophic factors” are substances (e.g., peptides (neuropeptides), that stimulate the growth of neurons (e.g., sympathetic nerve cells, sensory nerve cells) and are typically responsible for the regulation, growth and survival of neurons (e.g., maintaining neurons during development and fully developed neurons). Neurotrophic factors also are capable of assisting in the regeneration of damaged neurons (e.g., assisting in the regrowth of a neuron's processes) in vivo and/or ex vivo. Examples of neurotrophic factors include nerve growth factor (NGF), brain-derived neurotrophic factor (BDNF), novel neurotrophin-1 (NNT1), neurotrophin-3 (NT-3), and neurotrophin-4 (NT-4), neurotrophin 4/5 (NT 4/5), insulin-like nerve growth factor (IGF such as IGF-1, IGF-2), glial fibrillary acidic protein (GFAP), laminin B2, ciliary nerve growth factor (ciliary neurotrophic factor (CNTF)), leukemia inhibitory factor (LIF) and S100.

“Angiogenic factors” or “pro-angiogenic factors” are substances (e.g., polypeptide, lipid) that causes the growth of new blood vessels. Examples of angiogenic factors include vascular endothelial growth factor (VEGF), von Willebrandt Factor (vWF), CD31, acidic and basic fibroblast growth factor, angiogenin, and transforming growth factors alpha and beta.

As used herein a “neural conduit” or “neurotrophic conduit” refers to a conduit (bioconduit) or passageway that is capable of generating or regenerating neural tissue when implanted in an individual. That is, the neural conduit can facilitate or propagate nerve (neural tissue) generation or regeneration when implanted in vivo. As is apparent to those of skill in the art, neural tissue is composed of neurons that receive, transmit and conduct impulses in the nervous system of an individual.

In particular aspects of the invention, a neural conduit is generated by introducing cells that enhance nerve generation into an isolated, naturally occurring “epineural sheath”. Typically, nerve fibers are wrapped in a connective tissue called the endoneurium. Groups of fibers surrounded by their endoneurium are arranged in bundles called fascicles, and each fascicle is wrapped in connective tissue called the perineurium. The outermost covering around the entire nerve is the epineurium. As used herein an (one or more) “epineural sheath” is an (one or more) epineurium of a (one or more) nerve. As described herein, an isolated, naturally occurring epineurium sheath is used in the methods of the invention. A “naturally occurring” epineural sheath refers to an epineural sheath obtained from natural sources; that is, an epineural sheath that is not synthetic (non-synthetic). Epineural sheaths that are “isolated”, include pure (essentially pure) epineural sheaths, that have been separated away from molecules and other tissues (e.g., endoneurium, perineurium, fasicles, blood components, inflammatory molecules) of their source of origin (e.g., an individual; an isolated nerve), and include epineural sheaths obtained by methods described herein or other suitable methods.

The epineural sheath can be obtained from a variety of nerves, such as nerves from invertebrates, vertebrates or a combination thereof. In one aspect, the naturally occurring, isolated epineural sheath is obtained from (isolated from) a mammalian nerve such as a nerve of primate (e.g., human), porcine, canine, feline, bovine, and/or murine origin. In other aspects, the epinueral sheath is an autologous epineural sheath, an allogenic epineural sheath, an isogenic epineural sheath, a xenogenic epineural sheath or a combination thereof. In a particular embodiment, the epineural sheath is obtained from a cadaver (e.g., a human cadaver).

In addition, the epineural sheath can be obtained from a variety of type of nerves, such as from a sensory nerve and/or a motor nerve. For example, although not necessary, in embodiments in which the neural conduit generated is used to repair a nerve gap in a sensory nerve, the epineural sheath can be obtained from a sensory nerve (e.g., from a sensory nerve that is the same as, similar to or different from, the sensory nerve that is being repaired); and in embodiments in which the neural conduit generated is used to repair a nerve gap in a motor nerve, the epineural sheath is obtained from a motor nerve (e.g., from a motor nerve that is the same as, similar to or different from, the motor nerve that is being repaired).

As will be apparent to one of skill in the art, all or a portion of a naturally occurring, isolated epineural sheath can take a variety of shapes for use in the methods of the invention, and the shape will depend upon a variety of factors, such as the properties of the nerve that is to be repaired (e.g., nerve type, nerve diameter, nerve length), the type of nerve injury and/or the condition of the individual (e.g., patient). For example, one or more epineural sheaths can be used as a tube (e.g., a tube having two free ends or lumens; a hollow tube), or one or more tubes can be longitudinally split and used as a flat rectangular sheath. In addition, one or more epineural sheaths can be formed into one or more strips, cords (e.g., twisted strips, plain or enriched with cells), patches, scaffolds (e.g., filled with cells, slow-releasing growth factor), pastes, powders (e.g., with a gel), putty(ies) or a combination thereof for use in the methods of the invention. As will be apparent to one of skill in the art, one or more of these forms can be achieved using one or more epineural sheaths (e.g., multiple epineural sheaths secured together, e.g., as a large sheet or secured together in multiple layers and filled with powder, gel and/or factors that enhance nerve growth and/or regeneration).

In particular, all or a portion of a naturally occurring, isolated epineural tube can be used. In another embodiment, one or more naturally occurring, isolated epineural tubes can be used in the methods. In another embodiment, one or more naturally occurring, isolated epineural tubes can be split (e.g., longitudinally) and used as a (e.g., flat) rectangular sheath in the methods. In embodiments in which two or more naturally occurring, isolated epineural tubes are split longitudinally thereby producing two or more rectangular sheaths, the two or more rectangular sheaths can be used to make a large rectangular sheath or placed in layers. In yet another embodiment, the epineural tube can be split (e.g., longitudinally) into one or more strips, and the epineural strips can be used in the methods described herein.

As is apparent to one of skill in the art, different lengths and diameters of epineural sheaths may be used in the methods of the invention, and will depend upon a variety of factors, such as the properties of the nerve that is to be repaired (e.g., nerve type, nerve diameter), the type of neural injury (e.g., the dimensions, such as length and width, of a nerve gap) and/or the condition of the individual. In some embodiments, the epineural sheath can be from about 1 mm to about 150 cm in length. In other embodiments, the epineural sheath can be from about 1 cm to about 150 cm in length, about 10 cm to about 140 cm, about 20 cm to about 130 cm, about 30 cm to about 120 cm, about 40 cm to about 110 cm, about 50 cm to about 100 cm, about 60 cm to about 90 cm, and about 70 cm to about 80 cm. For example, the epineural sheath can be about 1 cm, 10 cm, 20 cm, 30 cm, 40 cm, 50 cm, 60 cm, 70 cm, 80 cm, 90 cm, 100 cm, 110 cm, 120 cm, 130 cm, 140 cm, 150 cm, 160 cm, 170 cm, 180 cm, 190 cm or 200 cm.

In other embodiments, the epineural sheath can be from about 1 cm to about 10 cm in width. For example, the epineural sheath can have tube diameters of about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm; lengths of about 1 cm, 20 cm, 30 cm, 40 cm, 50 cm, 60 cm, 70 cm, 80 cm, 90 cm, 100 cm, 110 cm, 120 cm, 130 cm, 140 cm, 150 cm; and patch sizes of about 1 cm²×100 cm² such as 2×2 cm, 3×3 cm, 4×4 cm, 5×5 cm, 6×6 cm 7×7 cm, 8×8 cm, 9×9 cm, 10×10 cm, 20×20 cm, 30×30 cm, 40×40 cm, 50×50 cm, 60×60 cm, 70×70 cm, 80×80 cm, 90×90 cm, 100 cm×100 cm, etc. Epineural sheaths in forms such as tubes and patches can be linked or custom designed (cut from whole sheath etc.).

Methods for obtaining or harvesting isolated, naturally occurring epineural sheaths are provided herein, and are known to those of skill in the art (e.g., see PCT/US2009/039258; WO2009/124170; Attorney Docket No. 3786.1032-001, which is incorporated herein by reference in its entirety). In addition, preservation methods to reduce immunogenicity for allografts and to keep stored epineural grafts for off shelf use and banking following methods are also provided herein. After harvesting, cyopreserved, cold stored, or lyophilized epineural sheaths can be used as different lengths, sizes, and widths.

Examples of methods for harvesting an isolated, naturally occurring epineural sheath from the sciatic nerve are provided herein. As will be apparent to one of skill in the art, other methods can be used to harvest an isolated, naturally occurring epineural sheath from other sources using routine skills. In one embodiment, the access to the peripheral nerve (e.g., sciatic nerve) is made by skin incision and subcutaneous tissue dissection down to the anatomical location of the nerve. At this level the sciatic nerve is cleared of all surrounding tissues by blunt dissection as far proximally as the sacral plexus and as far distally as its division into the terminal nerve branches. All collateral branches arising from the sciatic nerve throughout its length can be detached and used separately to create an epineurial sheath tubular grafts of different size diameters and lengths.

At this point the sciatic nerve is ready to be dissected out. The nerve is transected as proximal as is feasible at its origin from the sacral plexus, and then transected distally where the nerve divides into its terminal components, at the level of insertion into the muscle.

Depending upon the area of nerve harvest, the nerve can then be suspended on either a straight driver/irrigator with round tip (e.g., 30 gauge×25 mm depending on nerve diameter—the driver diameter is typically smaller than nerve diameter), on a curved/hook finished driver/irrigator, or on a screwdriver type of irrigator. The irrigator can be filled with chilled solution (either cryopreservation solution for long term storage, or nerve culture medium or combination of both—depending on the fate of graft) and kept moist on the dissection board by soaking it with 0.9% sodium chloride.

Under microscope or loop magnification the axons can then gently be teased from its epineural sheath with the use of circular motion of driver/irrigator and jeweler fine forceps pulling the sheath away from the axons and driver in the “devaginating maneuver”, so that the axon fibers are pulled from the distal end whilst the epineural sheath is held from the proximal end on the driver/irrigator. Once all axons, the perineurium and the endoneurium are removed the intact, clear epineural sheath can be irrigated and left as a product of this process and is then inspected for integrity.

The epineural sheath of autologous, allogenic, xenogenic or isogenic origin can be harvested in the form of a full sheath, sheath/strips, and/or conduit (e.g., tube) and the neural conduit generated therefrom can be applied, for example, to fill nerve defects; to cover nerves and neural tissues protect them from scarring after surgery; to provide nerve guidance at long gap distances or for coverage of spinal nerves; as dura (e.g., for dura tear) of the spinal cord as well as a patch for coverage of dural and brain defects; and as a combination of these applications for different types of applications in peripheral nerve surgery, plastic surgery, orthopedics, vascular surgery, spine and neurosurgery.

As described herein, one or more agents that enhance (promote) nerve generation or regeneration is introduced into (onto) an isolated naturally occurring epineural sheath. As shown herein, such agents include saline, all or portions of another (one or more) epineural sheath (e.g., one or more epineural strips, epineural powder), cells and combinations thereof. In a particular embodiment, the one or more agents is cells that enhance (promote) nerve generation or regeneration are introduced into an isolated naturally occurring epineural sheath to produce a combination. As is apparent to one of skill in the art, cells that enhance nerve generation include stromal cells (e.g., bone marrow stromal cells (BMSC)), mesenchymal stromal cells, or a combination thereof (e.g., chimeric cells). As used herein, a “chimeric cell” refers to a cell which is a fusion of one or more autologous, allogenic, or isogenic cells with one or more autologous, allogenic, or isogenic cells. The fused cells can be the same (e.g., one or more BMSC fused with one or more BMSC), similar (e.g., one or more BMSC fused with one or more mesemchymal stromal cells) or different (e.g., one or more BMSC fused with one or more dendritic cells) type of cell. In a particular embodiment, the chimeric cell is a donor cell (e.g., a donor origin bone marrow progenitor such as a CD90 cell) fused with a recipient cell which is the same type of cell as the donor cell (e.g., a donor origin bone marrow progenitor such as a CD90 cell).

As is apparent to those of skill in the art, the cells can also comprise a label for detection/visualization either ex vivo or in vivo. For example, cells can be labeled with PKH-26, which stays visible for over 100 days.

The epineural sheath and/or cells that enhance nerve regeneration can be autologous (obtained from the individual), isogenic (obtained from an individual with an identical genotype), allogeneic (obtained from different individual of same species), xenogenic (obtained from different individual of different species) or a combination thereof to each other and/or to the individual into which the neural conduit will be introduced.

Although as shown herein it is not necessary for the generation of a neural conduit comprising neurotrophic factors and angiogenic factors, those of skill in the art will appreciate that the epineural sheath can be contacted with additional factors, including additional (exogenous) neurotrophic factors and angiogenic factors, in order to further enhance the generation of neurotrophic factors and angiogenic factors therein. Such factors also include additional cells that aid and/or enhance generation or regeneration of neural tissue. Examples of such cells include progenitor cells, stem cells (e.g., mesenchymal stem cells), bone marrow derived cells, dendritic cells, adipose (fat) cells, or chimeric cells. The cells can be autologous, allogenic, isogenic, xenogenic or chimeric cells. Examples of such factors include neurotropic and neurotrophic factors. Specific examples include nerve growth factors (NGF), vascular endothelial growth factor (VEGF), brain derived nerve growth factor (BDNGF), insulin-like nerve growth factor (INGF), glial fibrillary acidic protein (GFAP), laminin B2, cilliary nerve growth factor. In addition, pro-angiogenic factors such as VEGF and vWF, and cytokines such as IL-2, IL-3 and TGF-β can be used.

As one of skill will also appreciate, a variety of methods can be used to introduce or contact an epineural sheath with agents such as fillers to expand the volume of an epineural sheath, and/or cells that enhance nerve generation into the epineural sheath. For example, the introduction or contact can be performed using a syringe, a catheter, an infusion pump or a combination thereof. Depending on the conditions, the methods described herein can further comprise closing one or both ends of the neural conduit (e.g., closing one end of a neural tube) after the cells are introduced.

The epineural sheath and/or neural conduit is maintained under conditions in which neurotrophic factors, angiogenic factors, and/or neural tissue forms therein. A variety of conditions suitable for maintaining the epineural sheath and/or neural conduit for generation of neurotrophic factors, angiogenic factors, and/or nerve tissue therein will be apparent to those of skill in the art. In one aspect, such conditions comprise contacting the epineural sheath with saline, epineurium or a combination thereof, culturing the tube for about 3 days, 7 days, 14 days, 21 days, 28 days or 35 days in a cell culture medium at a temperature of about 37° C. in 5% CO₂.

The methods of generating the neural conduit described herein can further comprise detecting the presence of neurotrophic factors and/or angiogenic factors in the neural conduit. As will be appreciated by those of skill in the art, a variety of methods for detecting neurotrophic factors and/or angiogenic factors in the neural conduit are available. For example, the presence of neurotrophic factors (e.g., NGF, GFAP, S100) and/or angiogenic factors (e.g., VEGF, vWF, CD31) can be detected using immunohistochemical analysis for the detection of neurotrophic factors, pro-angiogenic factors or a combination thereof or electron microscopy for quantitative and qualitative evaluation of neurotrophic factors and/or angiogenic factors.

The neural conduit described herein can be used in vivo immediately after it is produced or stored for use at a later time. Thus, the methods can further comprise storing the neural conduits for use at a later time. For example, the neural conduits can be stored at 4° C. for about 1 hour, 4 hours, 8 hours, 16 hours, 24 hours, 48 hours or 72 hours. Alternatively, the neural conduits can be stored at −196° C. for about 4 days, 5, days, 6 days, 1 week, 2 weeks, 1 month, 3 months, 6 months, 9 months, 1 year, 2 years, 3 years, 4 years, or 5 years.

In a particular aspect of the invention, the method of generating the neural conduit ex vivo can further comprise transplanting the neural conduit into an individual in need thereof. For example, the individual can have a neural defect such as a nerve gap wherein the neural conduit (e.g., in the form of an epineural tube segment) will match the size of the gap (e.g., a length of about 1, 2, 3, 4, 5, 6, 7, 8, 9, 10, 11, 12, 13, 14, 15, 16, 17, 18, 19, 20 cm between a proximal nerve stump and a distal nerve stump, and a diameter of about 1, 2, 3, 5, 10, 15, 20, 25, 30 mm). The neural conduit can also be implanted in individuals having neural tissue that needs to be protected from exposure to, for example, the environment and/or inflammatory factors (e.g., a dural defect). Also, the neural conduit can be implanted in an individual after neuroma revision surgery to inhibit formation, or in an individual undergoing a decompression procedure.

In particular embodiments, the neural conduit can match the type of nerve to which it will be connected, which can be either purely sensory (e.g., sural nerve), purely motor (e.g. radial nerve), or mixed sensorimotor nerve type (e.g., median nerve). In these embodiments, the sensory tube will connect the sensory nerve stumps, motor nerve stumps, and sensorimotor stumps.

In another aspect, the invention is directed to neural conduits produced by the methods provided herein. In one embodiment, the invention is directed to a neural conduit comprising an isolated, naturally occurring epineural sheath (e.g., a no cell epineural sheath (NCEC); a no stromal cell epineural sheath (NSCES)). Such conduits can further comprise agents used to expand the volume of the epineural sheath such as saline, epineural strips and/or epineural powder. In another embodiment, the invention is directed to a neural conduit comprising an isolated, naturally occurring epineural sheath and cells which enhance neural regeneration (e.g., a stromal cell epineural sheath (SCEC)). In a particular embodiment, the epineural sheath of the neural conduit forms an epineural tube.

Another aspect of the invention is an article of manufacture. In one embodiment, the article of manufacture comprises one or more isolated naturally occurring epineural sheaths, a device for introducing agents (e.g., saline, epineural strips, epineural powder, cells) which enhance neural regeneration into the one or more epineural sheaths, and instructions for use thereof (e.g., production of a neural conduit). In a particular embodiment, the epineural sheath forms an epineural tube.

In another embodiment, the article of manufacture comprises one or more neural conduits, wherein each neural conduit comprises an isolated naturally occurring epineural tube and cells which enhance neural regeneration, and instructions for use thereof.

In yet another embodiment, the article of manufacture comprises a neural conduit which contains neurotrophic factors, angiogenic factors or a combination thereof, and instructions for use.

### EXEMPLIFICATION

### Example 1

### Methods

### Ex Vivo

In an attempt to find the optimal culture duration for NF expression, multiple Stromal Cell-Epineural Conduits (SCECs) were maintained and harvested at different time points using different combinations of allogenic vs. isogenic tubes and cells. 12 groups were created with 2 SCECs per group as follows:

Group 1 Isogenic Tube/Isogenic BMSCs 3 day culture

Group 2 Isogenic Tube/Isogenic BMSCs 7 day culture

Group 3 Isogenic Tube/Isogenic BMSCs 14 day culture

Group 4 Isogenic Tube/Isogenic BMSCs 21 day culture

Group 5 Allogenic Tube/Allogenic BMSCs 3 day culture

Group 6 Allogenic Tube/Allogenic BMSCs 7 day culture

Group 7 Allogenic Tube/Allogenic BMSCs 14 day culture

Group 8 Allogenic Tube/Allogenic BMSCs 21 day culture

Group 9 Allogenic Tube/Isogenic BMSCs 3 day culture

Group 10 Allogenic Tube/Isogenic BMSCs 7 day culture

Group 11 Allogenic Tube/Isogenic BMSCs 14 day culture

Group 12 Allogenic Tube/Isogenic BMSCs 21 day culture

### Bone Marrow Stromal Cell (BMSC) Isolation

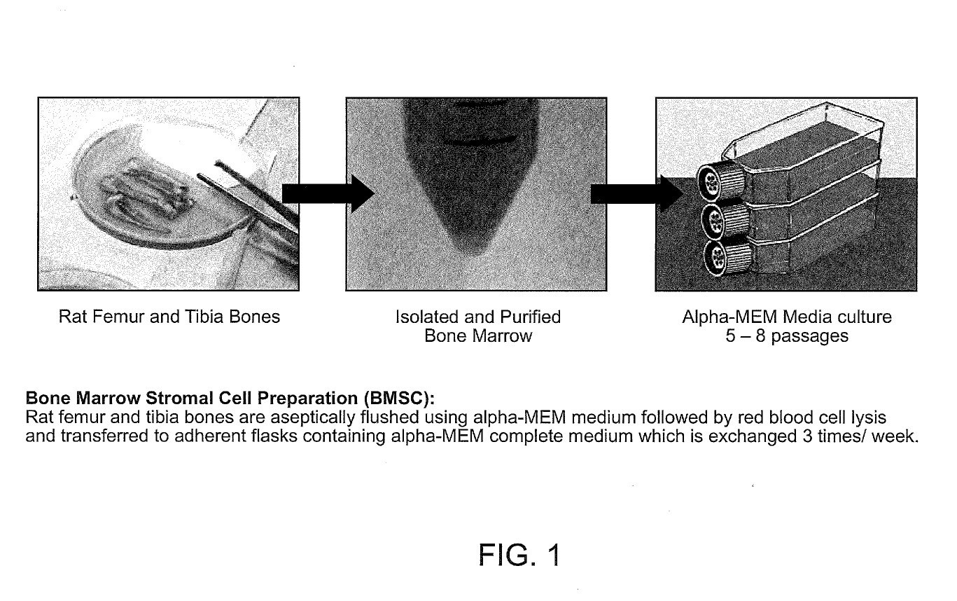

BMSCs were obtained from both adult isogenic and allogenic male rats (Harlan Sprague Dawley) and purified as previously described (Zurita, M and Vaquero, J., Neuroscience Letter, 402:51-56 (2006)). Fresh BMSCs were harvested aseptically from tibias and femurs of rats. Both ends of the bones were cut and the marrow was flushed with 10 ml of alpha-Minimum Essential (MEM) medium (Lerner Media Core Service). After centrifugation, the cell suspension was lysed with 0.85% NH₄Cl for 5 min, filtered through 70-mm nylon mesh, and re-suspended in alpha-MEM medium complete (alpha-MEM supplemented with 10% fetal bovine serum, 2 mM L-glutamine, 100 U/ml penicillin, 100 μg/ml streptomycin and 25 ng/ml amphotericin B) (see FIG. 1). The cells were placed in an adherent, 75 cm² flask and incubated at 37° C. in 5% CO₂ for 3 days. Non-adherent cells were removed by replacing the medium three times a week. After reaching confluence at 5 to 8 passages, BMSCs were enzymatically removed using 0.25% Trypsin and 1 mM % EDTA in PBS for 5 min, washed in alpha-MEM medium and prepared for membrane labeling using PKH-26 red dye. PKH-26 staining was performed in accordance to the manufacturer's instructions (Sigma Aldrich). Stromal cells were incubated with PKH-26 dye in Dilutent C buffer solution (Sigma Aldrich) at room temperature for 5 min. The reaction was stopped by incubation with 1% bovine serum albumin (BSA) in phosphate buffered saline (PBS) for 1 minute and complete alpha-MEM medium. Following the final wash, labeled stromal cells were prepared to a final concentration of 3×10⁶ cells and transplanted into either an isogenic or allogenic epineural sheath (FIG. 1). The surgical preparation of the epineural sheath and transplantation technique is described in detail below.

### Epineural Sheath Harvesting and Preparation

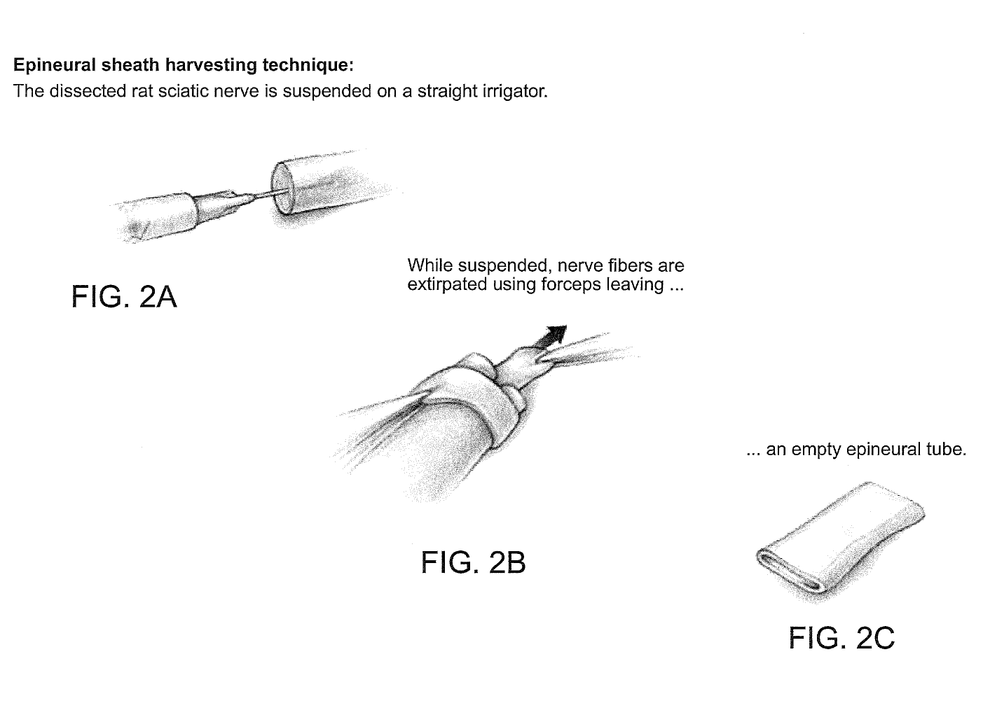

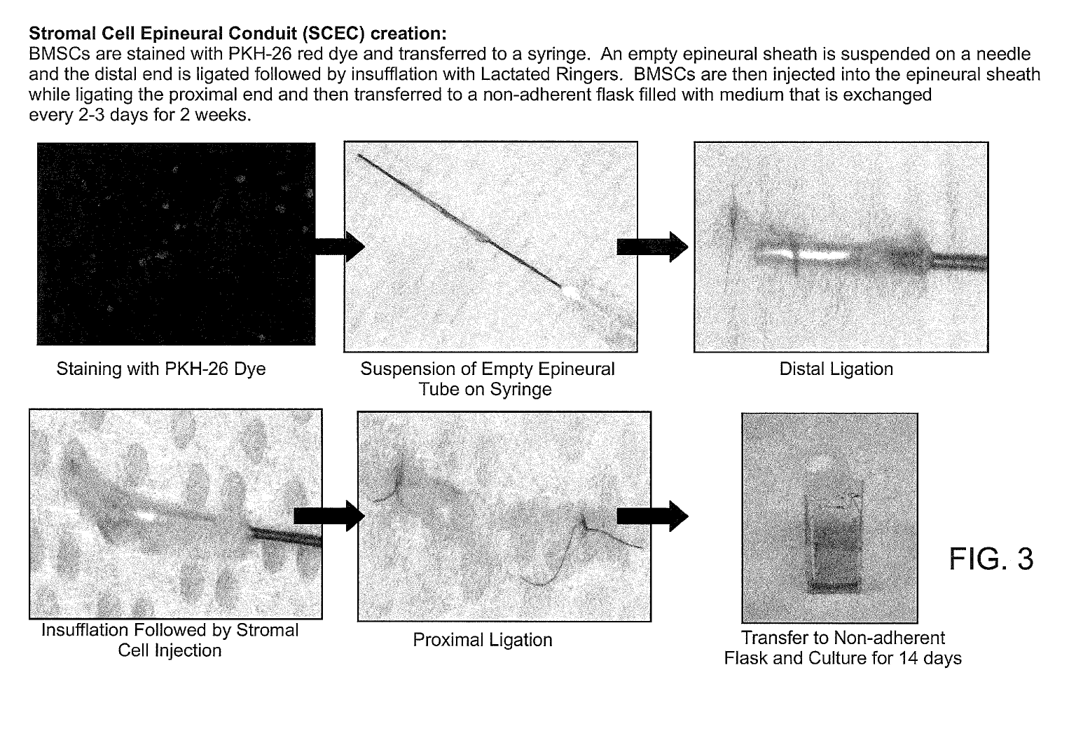

Epineural sheaths were prepared from the sciatic nerve of both isogenic and allogenic male rats. After an oblique skin incision was made in the right gluteal region, the sciatic nerve was exposed through a gluteal muscle-splitting incision and externally dissected to isolate a 20 mm segment of the nerve. The nerve was transected proximally and distally to obtain a 20 mm defect. The sciatic nerve was suspended on a straight irrigator (30 ga×1″ (25 mm) and while suspended the nerve fascicles were removed with the aid of fine surgical forceps from both the proximal and distal ends. This resulted in a 20 mm empty epineural tube conduit (FIG. 2). Prior to stromal cell injection, any visible branches, as well as the distal opening, were dissected and closed using 10-0 nylon suture to mitigate leakage. At this time, a small volume of lactated ringer's was injected to insufflate the collapsed epineurium (FIG. 3).

### Stromal Cell Injection Technique

The stromal cells were introduced using a 0.5 ml syringe by inserting the needle tip at the open proximal end and advancing towards the distal stump. Once the distal stump was reached, the stromal cells were uniformly injected while withdrawing the needle. As the needle tip was removed, a 10-0 suture was tightened around the proximal epineurium and used to prevent stromal cell leakage after syringe withdrawal. The stromal cell-epineural conduit (SCEC) was then placed in a non-adherent 25 cm² flask filled with alpha-MEM complete media. Half of the media was discarded and replaced every 2 days until it was removed for immunohistochemical evaluation at the aforementioned time points (FIG. 3).

### Immunohistochemical Analysis

At the specified time points, the SCEC was removed from the flask and snap-frozen in liquid nitrogen. Cut tissue slides were stained using mouse anti-rat monoclonal antibody for the neurotrophic factors NGF (H-20) (Santa Cruz Biotech), GFAP (2E1) (Santa Cruz Biotech), S100 (clone 4C4.9) (LabVision), and Laminin B (clone D18-2.2) (BD Pharmingen, CA) and for the pro-angiogenic factors VEGF (C1) and vWF (F8/86) (Santa Cruz Biotechnology, Inc) for 30 min. The binding of primary antibodies was detected using a rabbit anti-mouse immunoglobulin /FITC (DAKO, Carpinteria, Calif., USA) in accordance with the manufacturer's instructions. Slides were mounted in Vectashield® mounting medium with 4′-6-Diamidino-2-phenylindole (DAPI) and analyzed using a fluorescent microscope.

SCEC sections were also taken and immersion fixed in 3.5% glutaraldehyde, and embedded in Epon 812 (EMS, Ft. Washington, Pa.). Toluidine blue stain was used to stain 1-μm-thick cross-sections for light microscope evaluation of histological samples.

### In Vivo

Once the optimal culture duration was determined (e.g., in this aspect, about 14 days), the next step was to evaluate the SCEC's in vivo applications. 6 groups were created (n=3 per group) and evaluated at 6 and 12 weeks as follows after 14 days of culture:

Group 1 Isogenic Tube/Isogenic BMSCs 6 weeks

Group 2 Isogenic Tube/Isogenic BMSCs 12 weeks

Group 3 Allogenic Tube/Allogenic BMSCs 6 weeks

Group 4 Allogenic Tube/Allogenic BMSCs 12 weeks

Group 5 Allogenic Tube/Isogenic BMSCs 6 weeks

Group 6 Allogenic Tube/Isogenic BMSCs 12 weeks.

### SCEC Implantation

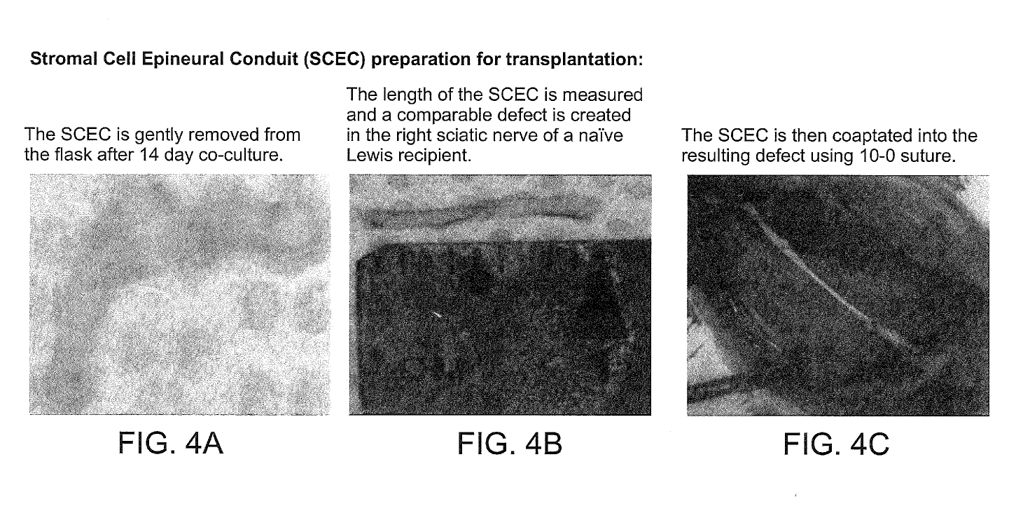

After two weeks, the right sciatic nerve of a naïve Lewis recipient rat was exposed as described above. Following exposure, a segment of sciatic nerve, comparable in length to the SCEC (FIG. 2B; ˜15-20 mm), was transected. The resulting defect was bridged by coaptating our previously prepared SCEC to the proximal and distal nerve stumps of our recipient rat (FIG. 4). All coaptations were performed using 10-0 nylon suture. Next, the gluteal muscles were reattached and the skin was closed primarily with 4-0 running vicryl sutures after achieving hemostasis. After the operation, the animals were allowed to walk freely in cages.

### Functional Assessment

Functional assessments were performed weekly using Pin-Prick (sensory recovery) and Toe-Spread (motor recovery). Videos were also taken to assess gait and walking.

### Pin-Prick

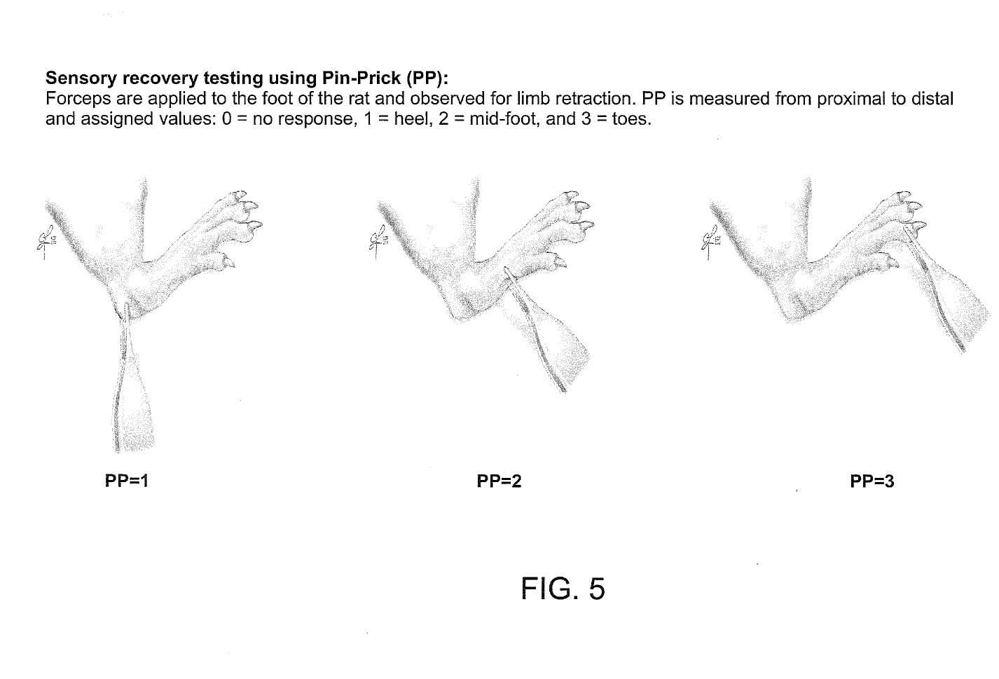

The pinprick test was used for evaluation of sensory recovery. A mild pinching stimulus was applied with forceps to the skin of the hind limb of the rat, from the toe to the knee joint level, until a withdrawal from the painful stimulus was elicited. The return of sensation was graded between 0 and 3 in the following manner: grade 0=no sensation, grade 1=presence of withdrawal response above the ankle, grade 2=presence of withdrawal response distal to the ankle in the heel/plantar region and grade 3=a withdrawal response from stimulus to the metatarsal region (FIG. 5).

### Toe-Spread

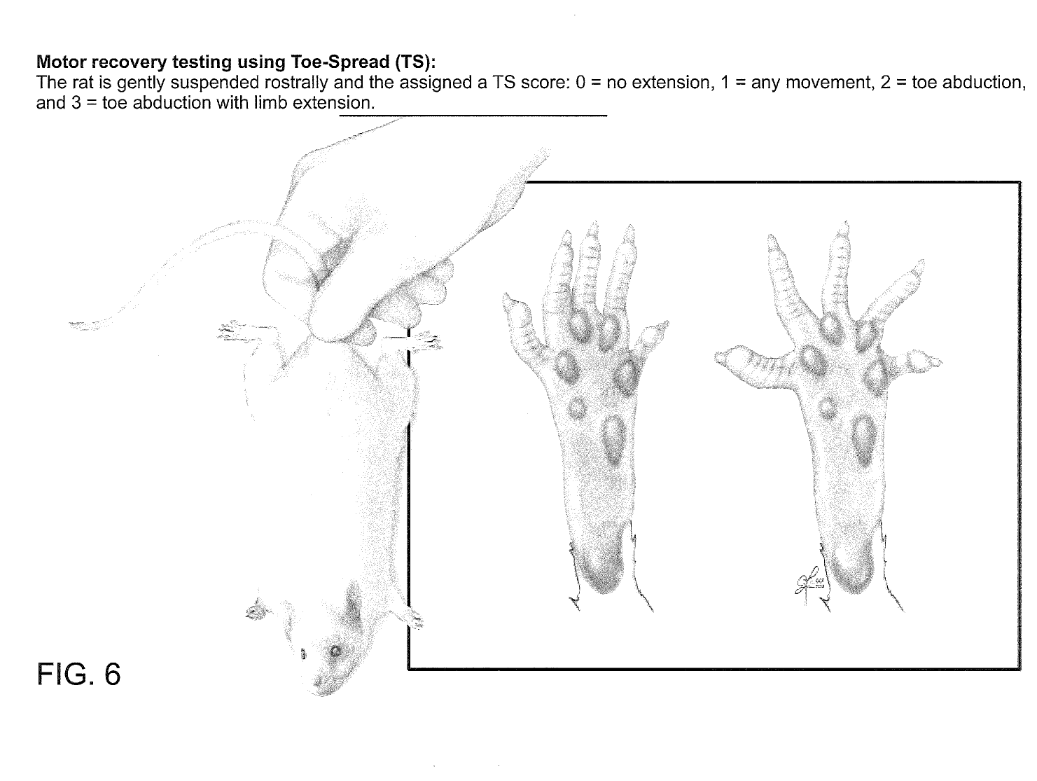

The toe spread test was used for evaluation of motor recovery. In the uninjured hind limb, the rat extends and abducts the toes when suspended by the tail. The toe spread was graded from 0 to 3 in the following manner: no toe movement=grade 0, any sign of toe movement=grade 1, toe abduction=grade 2, and toe abduction with extension=grade 3 (FIG. 6).

### Electrophysiological Assessment

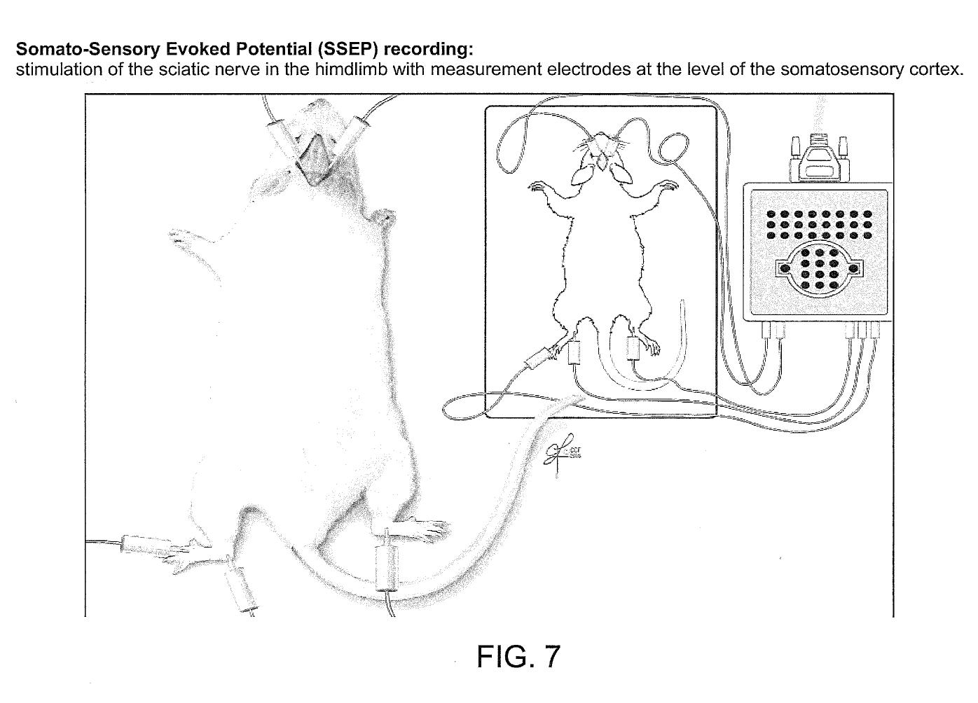

Sensory recovery in each group was evaluated by somatosensory evoked potential (SSEP) at 6 and 12 weeks using a Bio-logic A-PAC 486 computer system (Bio-logic Systems Corporation; Chicago, Ill.). Bandpass filter settings of 30 to 1,500 Hz were used. The gain setting was placed at 3,000. Repetition rate of the stimulus was set at 2.7 stimuli per second. A stimulus duration of 200 msec was used. Stimulation intensity was increased until a brisk motor twitch of the hind foot was obtained. Each response was replicated at least once. A display of a 100 msec window was used. After induction of anesthesia by pentobarbital (40 mg per kilogram intra-peritoneal), the stimulating electrodes (anode, cathode) were placed subcutaneously in the dorsum of the foot and the Achilles tendon on the operated right side to be evaluated, and the ground electrode was placed subcutaneously in the Achilles tendon of the contralateral foot. Next, a 2-cm sagittal incision was made on the scalp, and the cranium was exposed sub-periosteally. Detecting intracranial electrodes were placed through bilateral parietal burr holes, which were created with a hand-held drill. During control experiments, approximately three sets of 250 averages were obtained for baseline values. In these SSEP measurements, the waveform morphology consisted of a series of negative and positive potentials. The N1 latency was marked as the time point when the first upward deflection (negativity) was seen in the cortical channel This is followed typically by a prominent downward deflection (positive) called the P1 response. The later upward deflection (negative) following the P1 potential was labeled as the N2 potential. Because the P1 and N2 waveforms are the most robust and consistent potentials, these latencies were used to compare sensory recovery between different treatment groups. Wave amplitudes were also measured as a means of assessing the degree of axonal regeneration. Additionally from these values we were able to extrapolate P1% and N2% values as a means of comparison between operated and non-operated limbs (FIG. 7).

### Histomorphometric Analysis

After assessment of functional recovery and SSEP evaluation at the aforementioned time points, a 20 mm section including the tubule as well as the proximal and distal nerve stumps were excised, immersion fixed in 3.5% glutaraldehyde, and embedded in Epon 812 (EMS, Ft. Washington, Pa.). The contralateral sciatic nerve was also prepared in the same fashion to act as an intra-animal control. Toluidine blue stain was used to stain 1-μm-thick cross-sections for light microscope evaluation of histological samples. Three cross sections on the operated side and one cross section of the naïve nerve were processed. In total, four sections were evaluated and included: the proximal nerve stump and tubule (P), the middle tubule (M), the distal tubule and stump (D), and the un-operated/contralateral nerve (C). From each segment, six representative fields were chosen by an investigator blinded to the treatment group. Captured fields were non-overlapping, with each sample representing ˜5% of the cross sectional area of the entire nerve. Images of these nerve sections were taken by a digital camera (Kodak DC120 zoom digital camera; Kodak, Rochester, N.Y.) mounted on an Olympus BH-2 light microscope. Each image was captured via an HP computer and evaluated using the software, Image Pro Plus (Media Cybernetics, Silver Springs, Md.) for the following parameters: total axon count, axonal density (axons/100 um²), number of myelinated and unmyelinated fibers, myelin thickness (um), myelin area (um²), axon area (um²), nerve area (um²), fiber diameter (um), and axon diameter (um).

### Immunohistochemical Analysis

Same as above.

### Muscle Denervation Atrophy

Muscle denervation atrophy was evaluated through comparative weights of the gastrocnemius wet muscle mass. The gastrocnemius muscle of both the operated and non-operated sides were carefully dissected from the surrounding tissue and weighed using a digital scale. A Gastrocnemius muscle index (GMI) was calculated by measuring the ratio of the operated to the un-operated muscle mass

### Results

### Ex Vivo

The presence of PKH positive cells (red) in the cultured epineurium confirmed the presence of BMSCs inside the epineural tube at days 3, 7 and 14 after culture was initiated. The potential of an ex vivo cultured allogenic epineurium (ACI) filled with isogenic stromal cells (Lew) to augment nerve regeneration was supported by the increased expression of Laminin B (green) as early as 3 days after culture and throughout the follow-up period up to 14 days. Neurotrophic factors (green) such as NGF, GFAP and S100 were detected at each time-point after epineurium/stromal cells co-culture. Double positive staining (orange) indicates that BMSCs may be capable of either surface expression or secretion of neurotrophic factors including NGF, GFAP and S100 (FIG. 7 a, b, c).

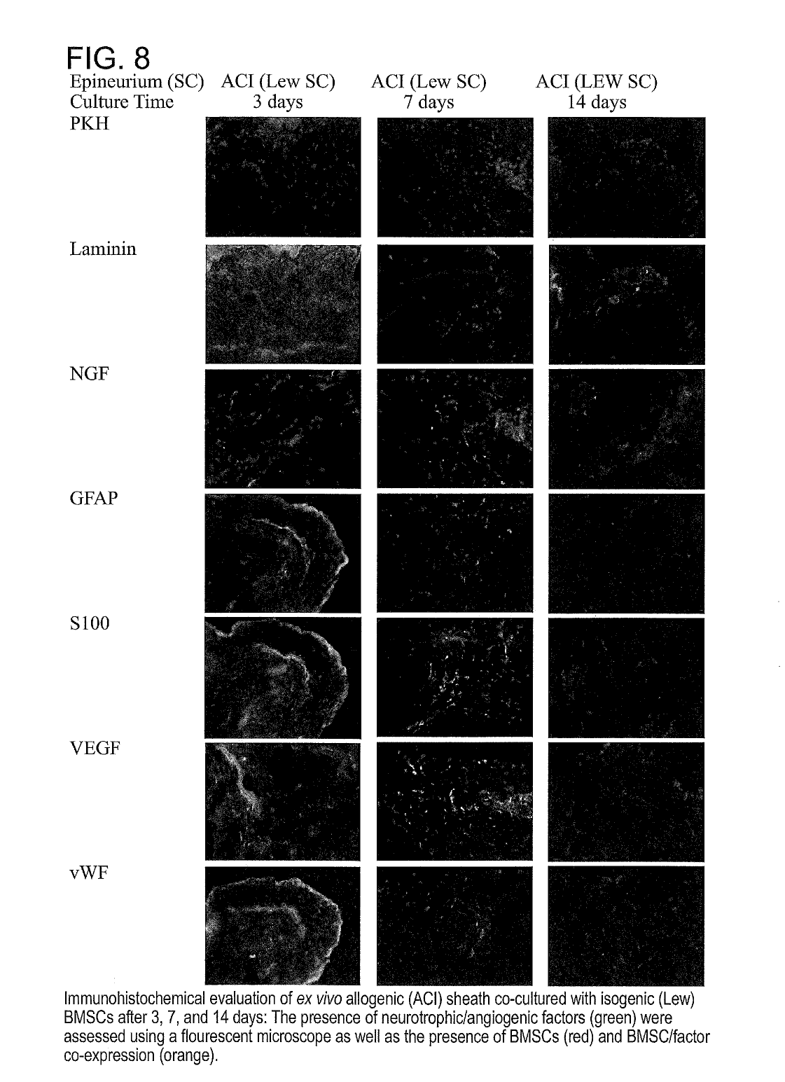

Immunostaining for the pro-angiogenic factors VEGF and vWF showed double positive cells for PKH/VEGF and for PKH/vWF. These results indicate that transplanted BMSCs have a pro-angiogenic capacity which likely enhances neural regeneration in vivo (FIG. 8).

The expression of both neurotrophic and pro-angiogenic factors following co-culture of an allogenic tube with isogenic stromal cells augments nerve regeneration following in vivo transplantation.

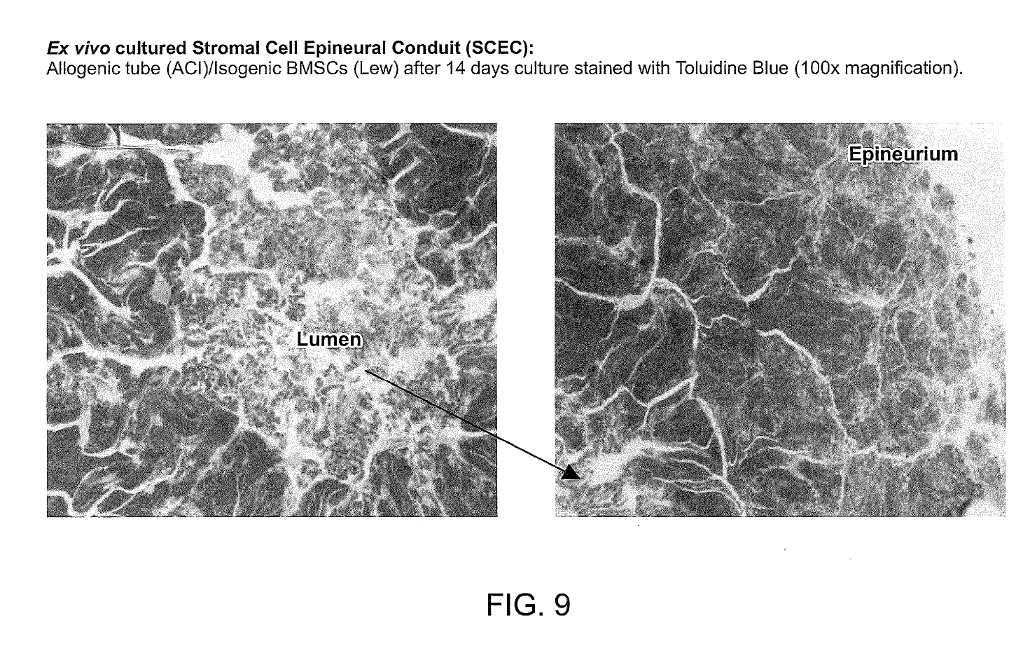

Toluidine Blue staining and evaluation under a light microscope revealed tissue devoid of axons with accumulation of staining at the periphery (FIG. 9).

In addition, the tube without cells filled with saline, epineural strips or epineural powder was the control group (epineural tube control) which was maintained under the same conditions as the epineural tube filed with cells. The media, time of culture etc. were the same for the control groups, the only difference was the lack of cells. The tube was also split open to create an epineural patch, and again the same conditions as described for the patch with cells and without cells (an epineural patch control) was also prepared. See FIG. 3 description.

Due to the fact that the epineural sheath is expressing constitutively Laminin B, the epineural sheath is likely a neuro-promoting sheath (e.g., epineural tube, epineural patch) and even without cell injection has inherent neuroregenerative and neuro-generating capacities.

### In Vivo

### Functional Data

4 SCECs were transplanted after 14 days of culture: 2 fully isogenic (isogenic tube and BMSCs), 1 fully allogenic (allogenic tube and BMSCs), and 1 mixed (allogenic tube and isogenic cells). Functional recovery as measured by Pin-Prick (PP) and Toe Spread (TS) after in vivo transplantation of a Stromal Cell Epineural Conduit (SCEC): Animals had different combinations of either Isogenic (Lew)/Allogenic Tubes with either Isogenic (Lew)/Allogenic BMSCs after 14 days culture ex vivo transplanted following sciatic nerve transfection. Sensory recovery was evaluated using Pin-Prick (PP) and motor recovery was measured using Toe Spread (TS) on a scale from 0-3.

The functional results were very promising demonstrating maximal sensory recovery after one week in 2 animals as measured by Pin-Prick (PP=3), 3 animals at week 2, and all animals at week 5. Motor recovery as measured by Toe-Spread has also been very robust with 3 out of 4 rats receiving a score of 2 by week 6. Functional results are shown in Table 1.

TABLE 1

| Animal | | | Week 1 | | Week 2 | | Week 3 | | Week 4 | | Week 5 | | Week 6 | |
|---|---|---|---|---|---|---|---|---|---|---|---|---|---|---|
| Number | Tube | BMSC | PP | TS | PP | TS | PP | TS | PP | TS | PP | TS | PP | TS |
| 1 | Allo | Iso | 3 | 1 | 3 | 1 | 3 | 1 | 3 | 1 | 3 | 1 | 3 | 2 |
| 2 | Iso | Iso | 3 | 0 | 3 | 0 | 3 | 1 | 3 | 1 | 3 | 1 | 3 | 2 |
| 3 | Iso | Iso | 2 | 0 | 2 | 1 | 2 | 1 | 2 | 1 | 3 | 1 | 3 | 1 |
| 4 | Allo | Allo | 1 | 1 | 3 | 1 | 3 | 2 | 3 | 2 | 3 | 2 | 3 | 2 |

### Histomorphometric Analysis

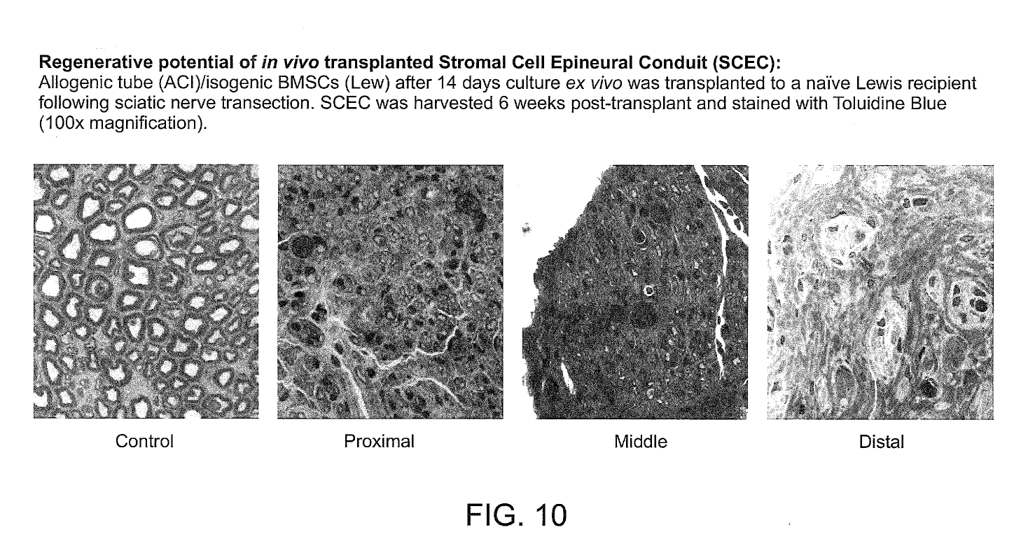

Currently, histomorphometric analysis has been performed on 1 Allogenic tube/Isogenic BMSC nerve 6 weeks post-transplantation. Myelinated axons and BMSCs can be identified (FIG. 10) with preliminary analysis demonstrating comparable myelination area and thickness, nerve area, and axonal density to isogenic tubes injected with either saline or isogenic BMSCs at the time of transection (Table 2).

TABLE 2

| | | ISO-EPI/ | ISO-EPI/ | | ISO-EPI/ | ISO-EPI/ | | ISO-EPI/ | ISO-EPI/ |
|---|---|---|---|---|---|---|---|---|---|
| | SCEC | ISO-SCs | ISO-SAL | SCEC | ISO-SCs | ISO-SAL | SCEC | ISO-SCs | ISO-SAL |
| Section | Proximal | Proximal | Proximal | Middle | Middle | Middle | Distal | Distal | Distal |
| Axon count | 924.00 | 772 | 1103.5 | 156.00 | 501 | 1147.5 | 114.0 | 847.33 | 605.5 |
| | | (277.19) | (335.88) | | (199.45) | (461.74) | | (330.85) | (495.68) |
| Axonal | 269.45 | 225.125 | 321.79 | 90.98 | 146.09 | 334.62 | 33.24 | 247.09 | 176.57 |
| Density | | (80.83) | (97.95) | | (58.16) | (134.65) | | (96.48) | (144.55) |
| (axons | | | | | | | | | |
| 100 um2) | | | | | | | | | |
| Myelinated | 813.00 | 734 | 814 | 138.00 | 461 | 1050 | 113.00 | 796 | 439 |
| Fibers | | (281.43) | (405.88) | | (192.72) | (567.10) | | (334.72) | (407.29) |
| Unmyelinated | 3.00 | 2.5 | 22.5 | 2.00 | 2.67 | 6.5 | 0.00 | 3 | 20.5 |
| Fibers | | (2.12) | (17.68) | | (1.53) | (6.36) | | (1) | (6.36) |
| Myelin | 0.57 | 0.70 | 0.59 | 0.69 | 0.64 | 0.79 | 0.73 | 0.69 | 0.60 |
| Thickness | | (0.12) | (0.04) | | (0.08) | (0.05) | | (0.05) | (0.12) |
| (um) | | | | | | | | | |
| Myelin Area | 3.30 | 4.54 | 4.09 | 4.89 | 3.98 | 6.24 | 4.52 | 4.55 | 4.21 |
| (um2) | | (1.58) | (0.02) | | (0.78) | (1.50) | | (0.58) | (1.04) |
| Axon Area | 1.12 | 1.50 | 1.98 | 1.49 | 1.24 | 2.18 | 0.93 | 1.43 | 202 |
| (um2) | | (0.61) | (0.46) | | (0.10) | (1.04) | | (0.26) | (0.18) |
| Nerve Area | 4.42 | 6.06 | 6.08 | 6.38 | 5.23 | 8.42 | 5.45 | 5.97 | 623 |
| (um2) | | (2.20) | (0.48) | | (0.88) | (2.54) | | (0.74) | (1.22) |
| Fiber | 2.31 | 2.63 | 2.64 | 2.89 | 2.45 | 3.15 | 2.63 | 2.67 | 265 |
| Diameter | | (0.45) | (0.08) | | (0.20) | (0.52) | | (0.14) | (0.31) |
| (um) | | | | | | | | | |
| Axon | 1.09 | 1.21 | 1.43 | 1.26 | 1.13 | 1.52 | 0.98 | 1.22 | 1.42 |
| Diameter | | (0.20) | (0.15) | | (0.06) | (0.36) | | (0.10) | (0.10) |
| (um) | | | | | | | | | |

Histomorphometric analysis 6 weeks after epineural tube transplantation: Comparison between transplanted allogenic tube (ACI) filled with isogenic stromal cells after 14 days culture (Stromal Cell Epineural Conduit—SCEC), isogenic (Lew) epineurium with immediate injection of isogenic (Lew) stromal cells (ISO-EPI/ISO-SCs), and isogenic (Lew) epineurium with immediate injection of saline (IS O-EPI/SAL) harvested at 6 weeks. Proximal, Middle, and Distal sections of transplanted epineuriem were stained with Toluidine Blue and viewed under a light microscope. Six images were taken per sample and analyzed using Image Pro-Plus.

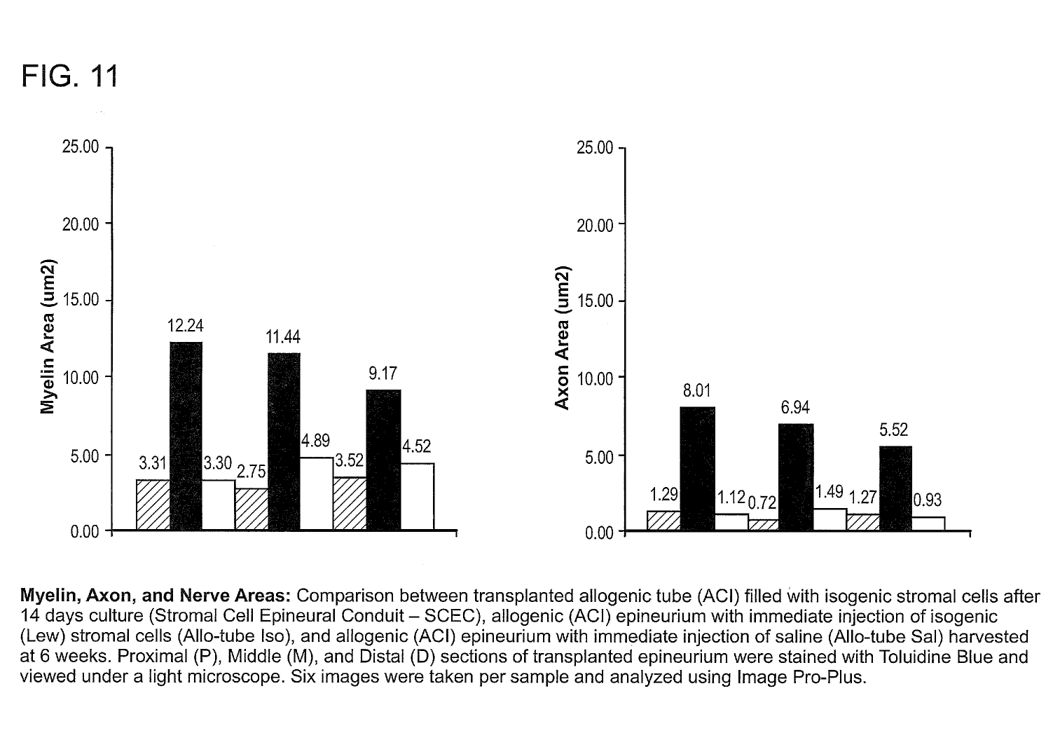

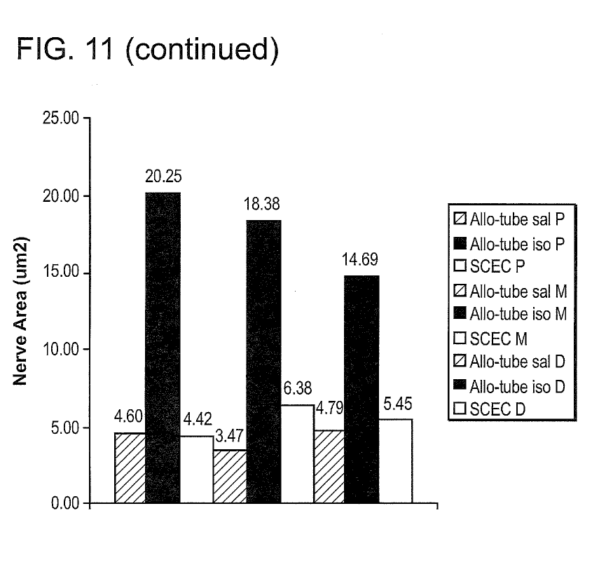

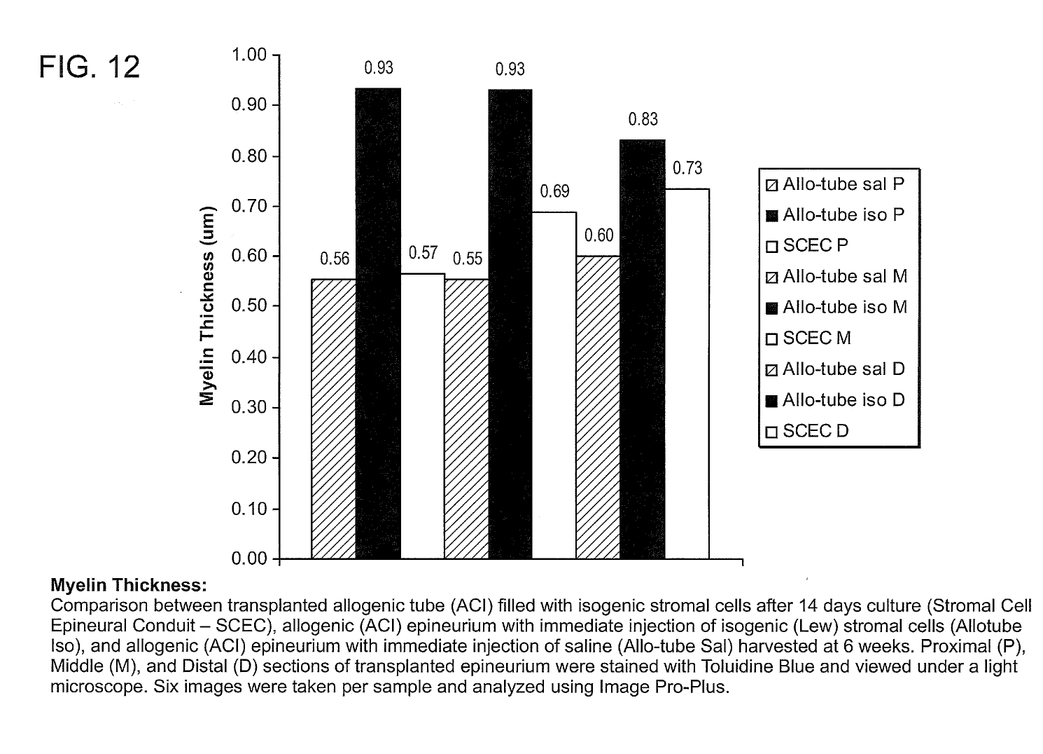

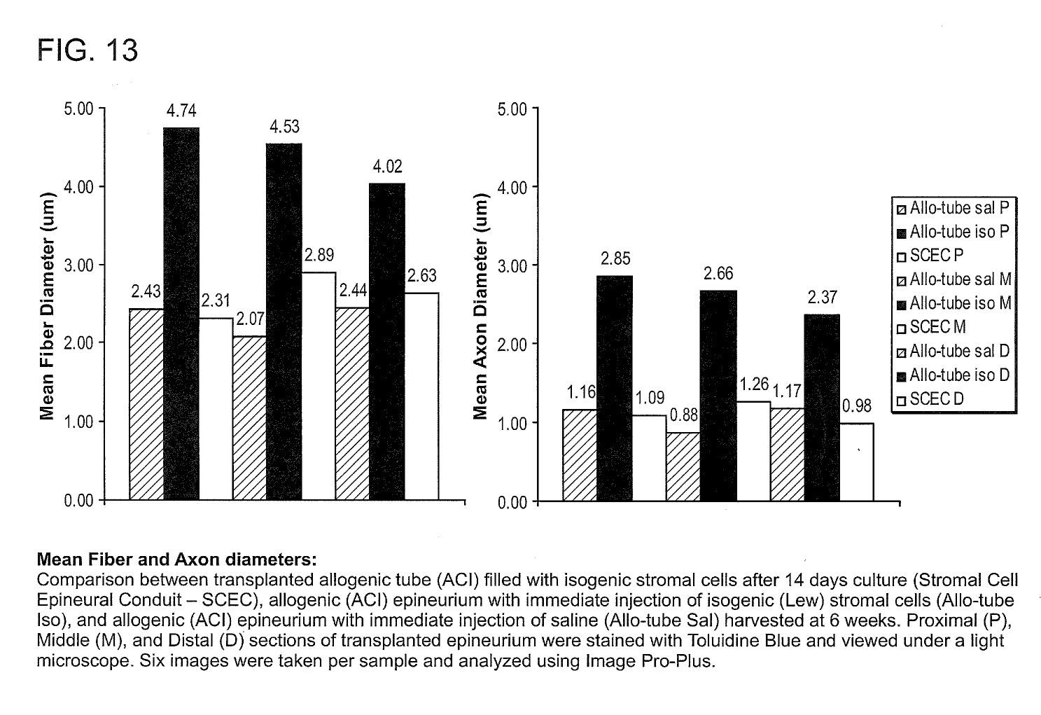

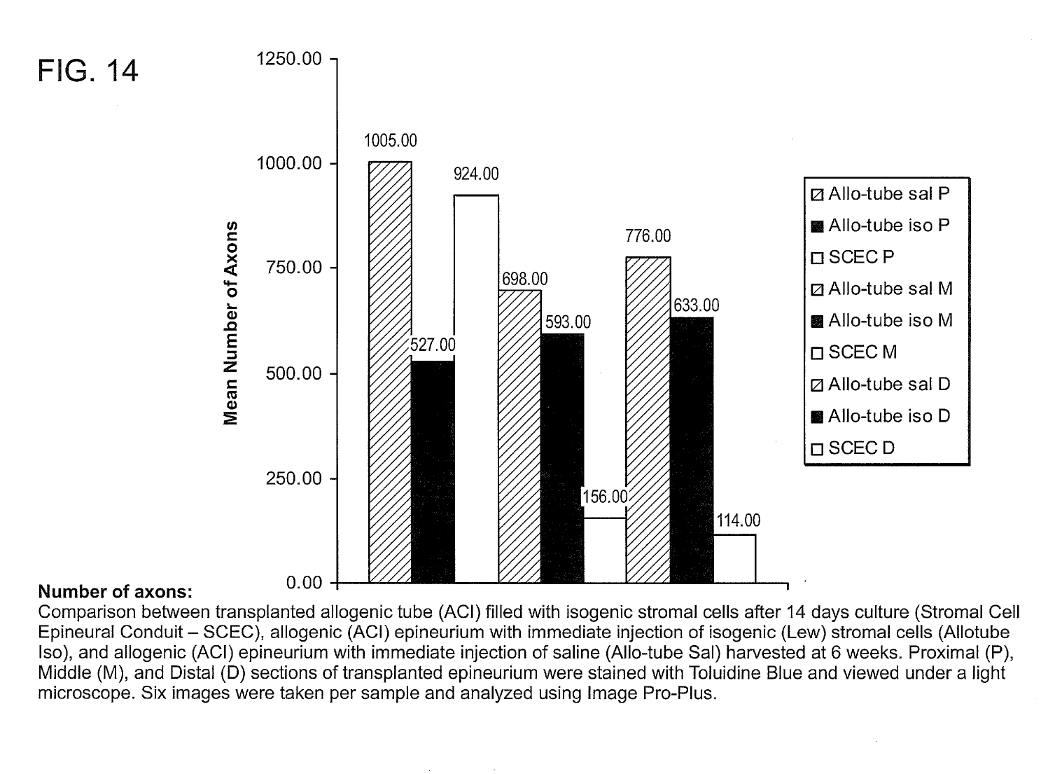

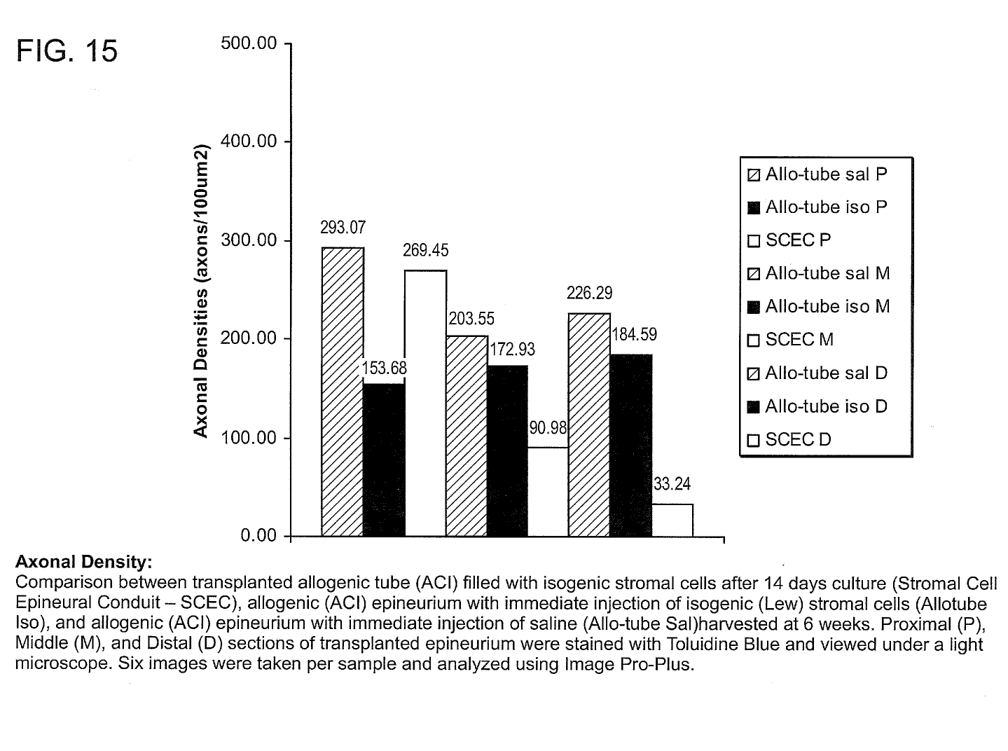

In FIGS. 11-15, measurements demonstrating good results with the use of allogenic (ACI) tubes augmented with isogenic (Lew) stromal cells when compared to allogenic (ACI) tubes filled with saline alone are shown for the following parameters: 1. myelin, axon, and nerve areas (FIG. 11) 2. myelin thickness (FIG. 12) 3. mean fiber and axon diameter (FIG. 13) 4. number of axons (FIG. 14) and 5. axonal density (FIG. 15).

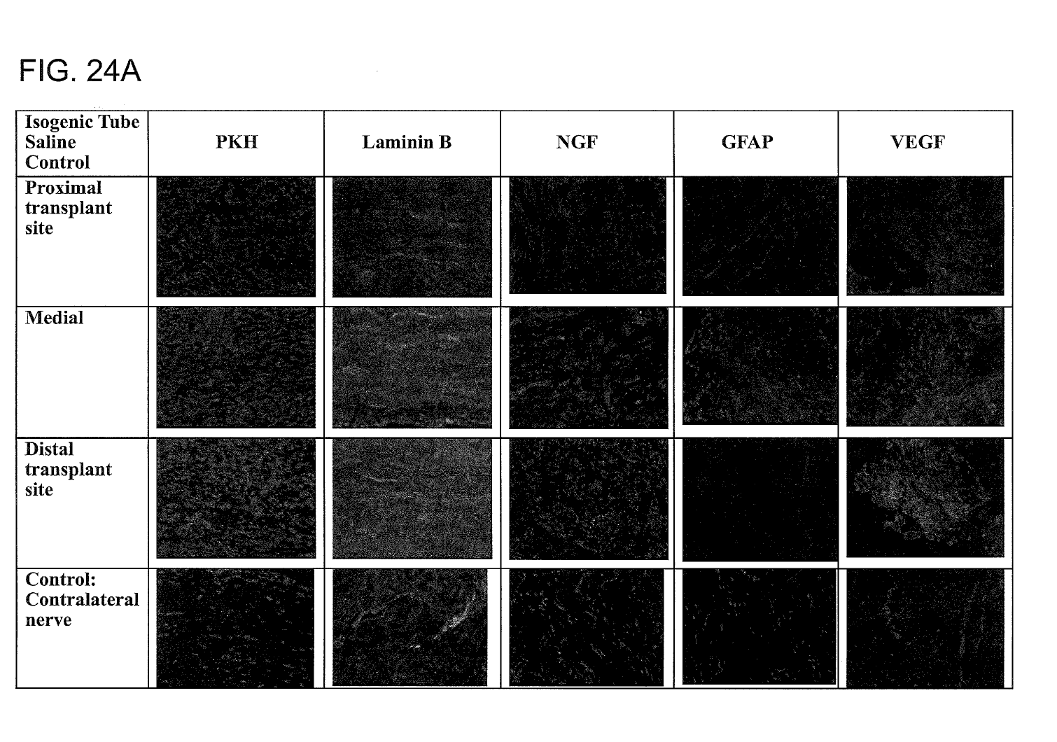

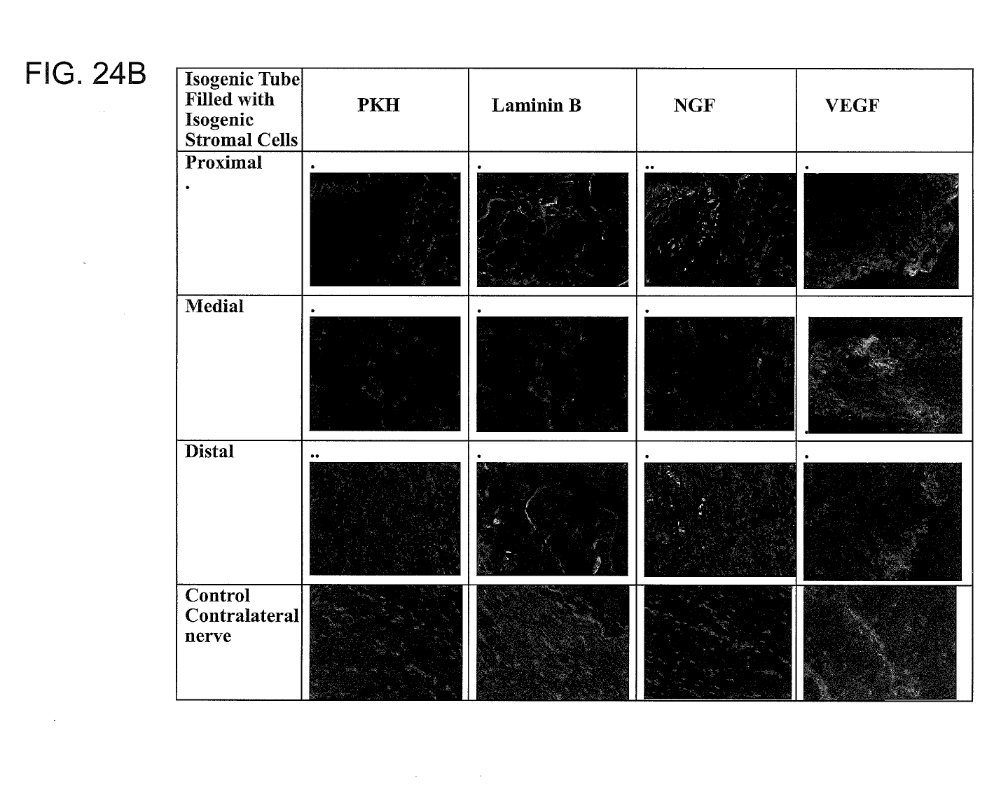

FIGS. 24A-24B show immunostaining data after engineered conduits were implanted into rats and evaluated 12 weeks after transplantation showing potential for nerve regeneration and expression of growth factors supporting nerve regeneration.

### Conclusions

Ex vivo induction of NF expression supports the epineural sheath's role as a viable and inherently neurotrophic conduit to be used following nerve injury. The expression of both neurotrophic (NGF, GFAP, and S100) and pro-angiogenic (VEGF and vWF) factors by BMSCs at all of the aforementioned time points highlights the role of the SCEC in enhancing in vivo neural regeneration.

In vivo results are also exciting with rapid recovery of both sensory and motor recovery as measured by functional tests (pin-prick and toe-spread). While the sensory recovery is comparable to ongoing studies that involve BMSC injection into a coaptated epineural sheath concomitantly, the speed of motor recovery is thus far superior using the SCEC paradigm. Early histomorphometric analyses demonstrated similar results, but given the robust expression of NF ex vivo and functional results, the SCEC will most likely yield better myelination and axon regeneration measurements.

### Example 2

### Monitoring Effects of Stromal Cells on Nerve Regeneration In Vitro

Expression of CD31, VEGF, GFAP, S100, WF, NGF and LAM 2 in rat sciatic nerve, rat sciatic nerve epineurial tube and cultured rat sciatic nerve epineurial tube filled with stromal cells was evaluated.

### Methods

Immunohistochemistry was applied to frozen sections prepared from rat's nerve, epineural tubes and stromal cells filled epineural tube cultured for 12 days. ACI and Lewis rats were used.

ELISA was used to measure level of NGF secreted into culture medium by stromal cells, by epineural tubes filled with saline and by epineural tubes filled with stromal cells.

### Results

### Immunohistochemistry

CD 31 was expressed inside of cultured epineural tube and co localized with dividing cells. It is likely that these dividing cells were derived from stromal cells. The CD 31 staining of sciatic nerve is associated with vasculature of epineurium.

The highest level of LAM2 seems to be associated with fresh sciatic nerve as compared with cultured epineural tube.

VEGF, GFAP, S100 were detected at low level in all samples analyzed.

NGF was not detected using this IHC staining.

The von Willebrandt Factor (vWF) is expressed in highest level in stromal cells filled cultured epineural tubes.

### ELISA

The rat β-NGF DuoSet ELISA Development System ((RnDSystems.com) was applied to measure level of NGF in culture media. The range of NGF detection in this assay is between 15 ng/mM and 1,000 pg/mL.

Media samples were collected from cultured stromal cells prepared from Levis rat bone marrow cells on days 3, 7, 18 and 20. Media from cultured epineural tubes (filled with saline or with Lewis rat stromal cells) were also collected form 3, 7 and 18 days old cultures.

### Results

NGF was not detected in Lewis rat cultured epineural tubes with saline.

NGF was detected in media of “older” cultures: at 18 and 20 days old culturing of Lewis stromal cells (143 pg/mL) and in 18 days old culturing Lewis rat epineural tubes filled with Lewis rat derived stromal cells (31 pg/mL).

The much lower level of NGF in epineural (“sausages”) than in stromal cells cultures observed in this experiment can be explained by smaller number of stromal cells in a “sausage” than in stromal cell cultures.

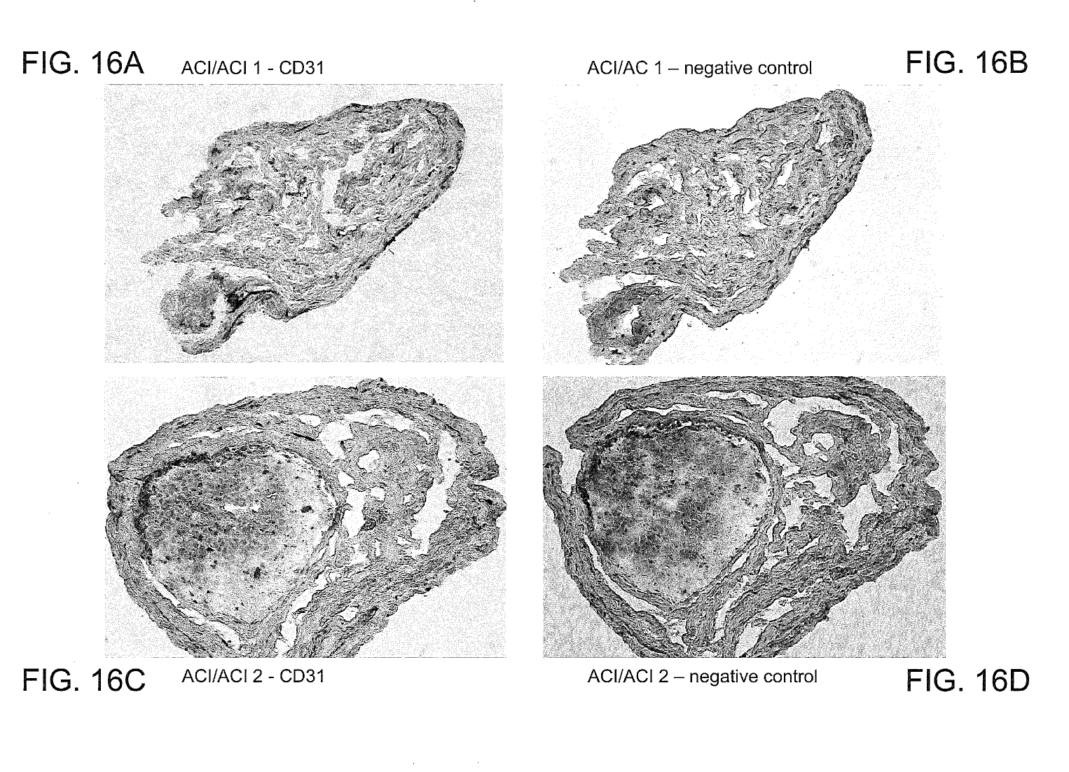

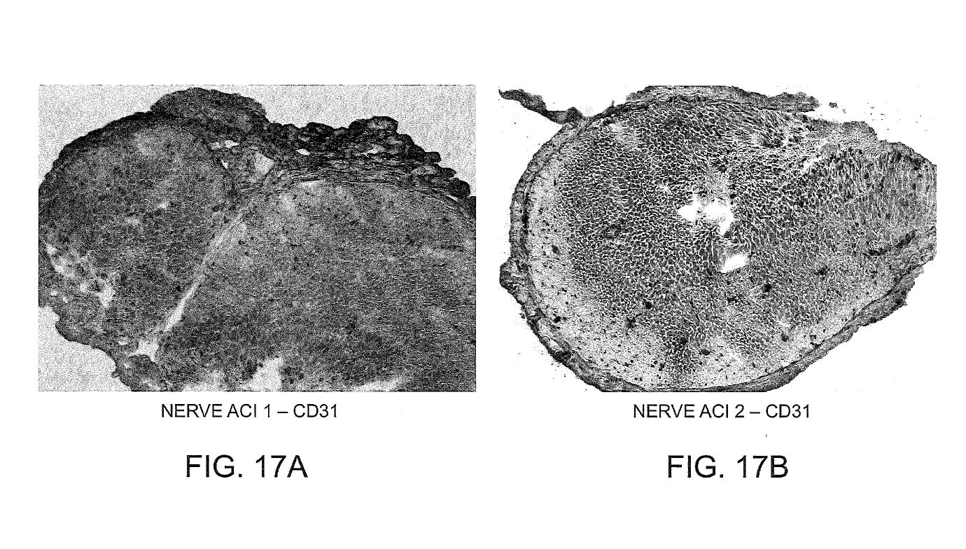

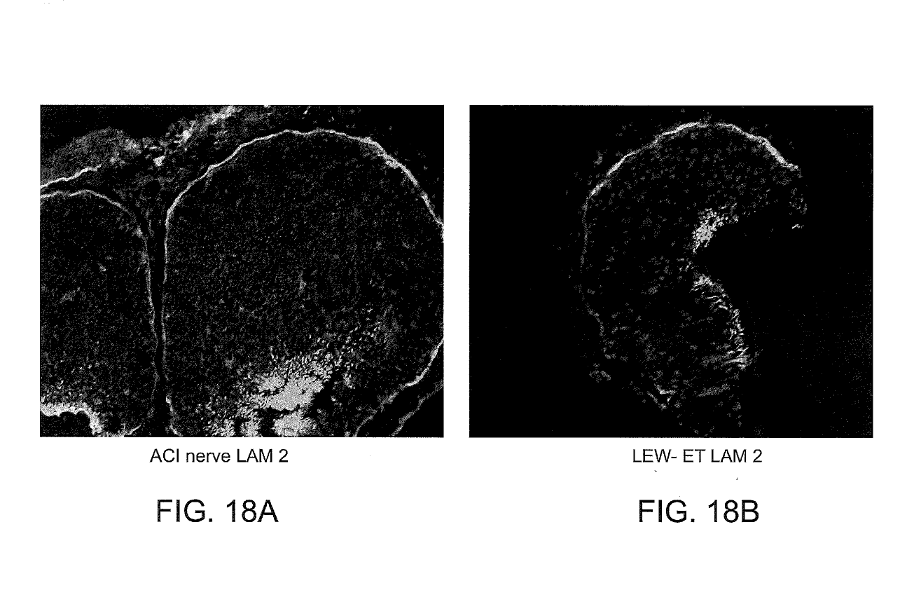

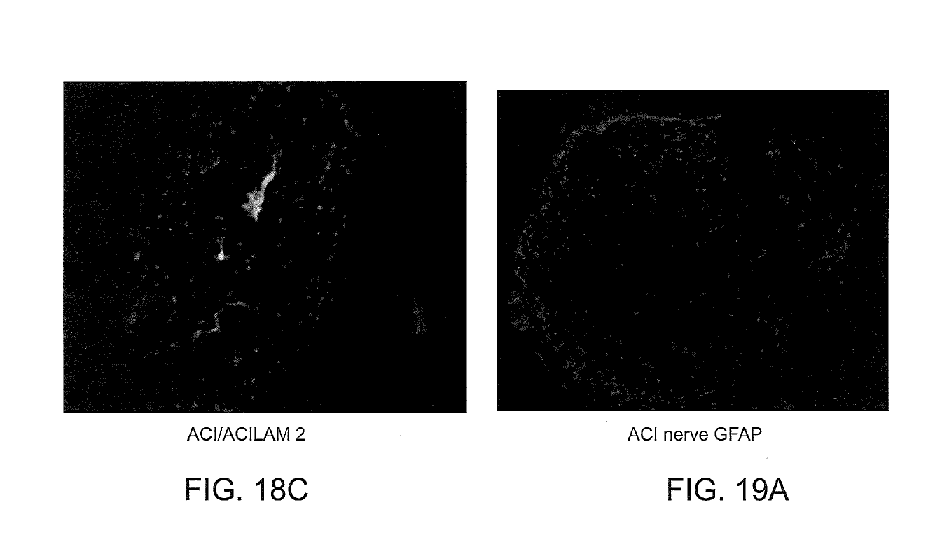

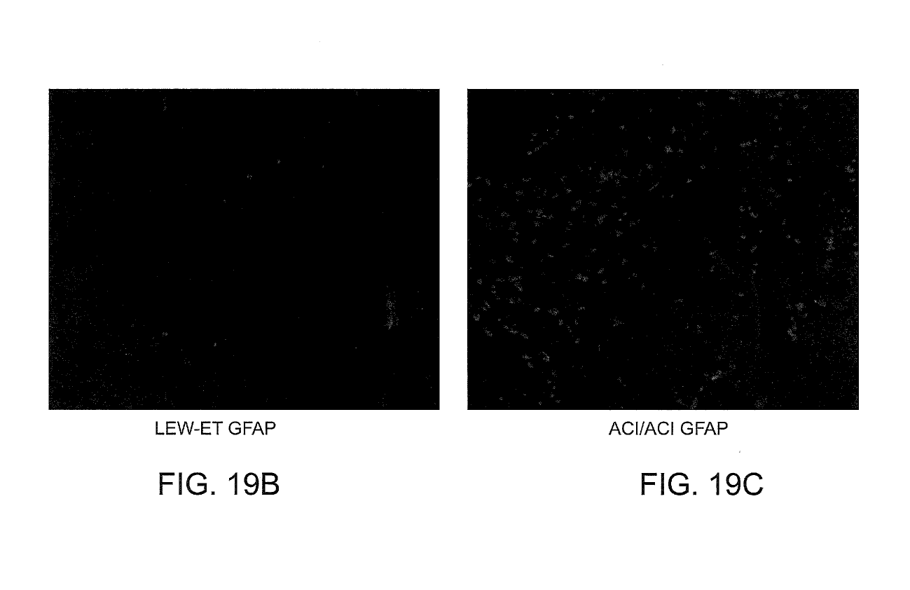

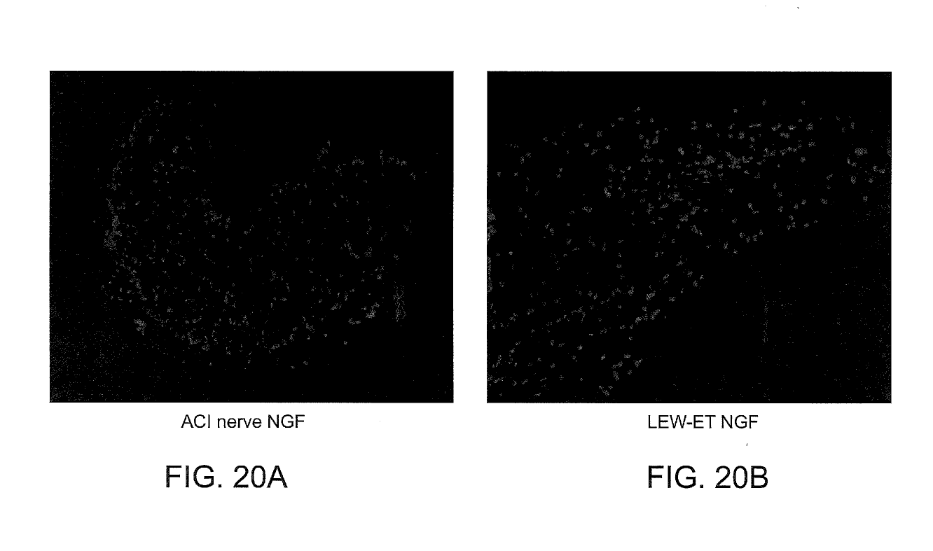

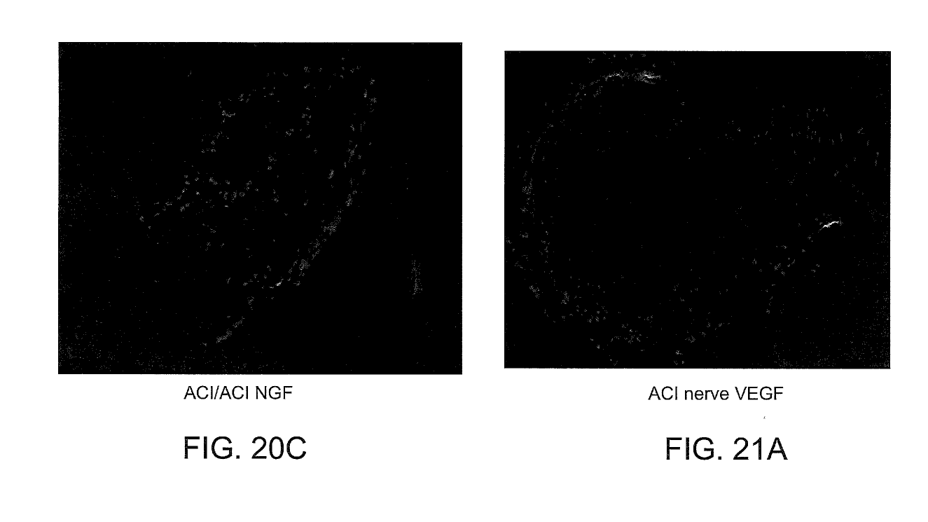

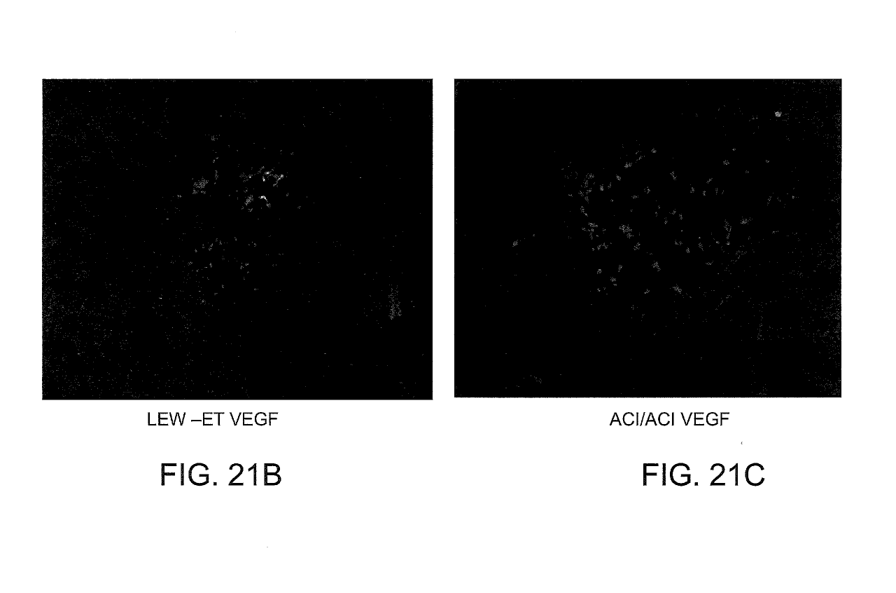

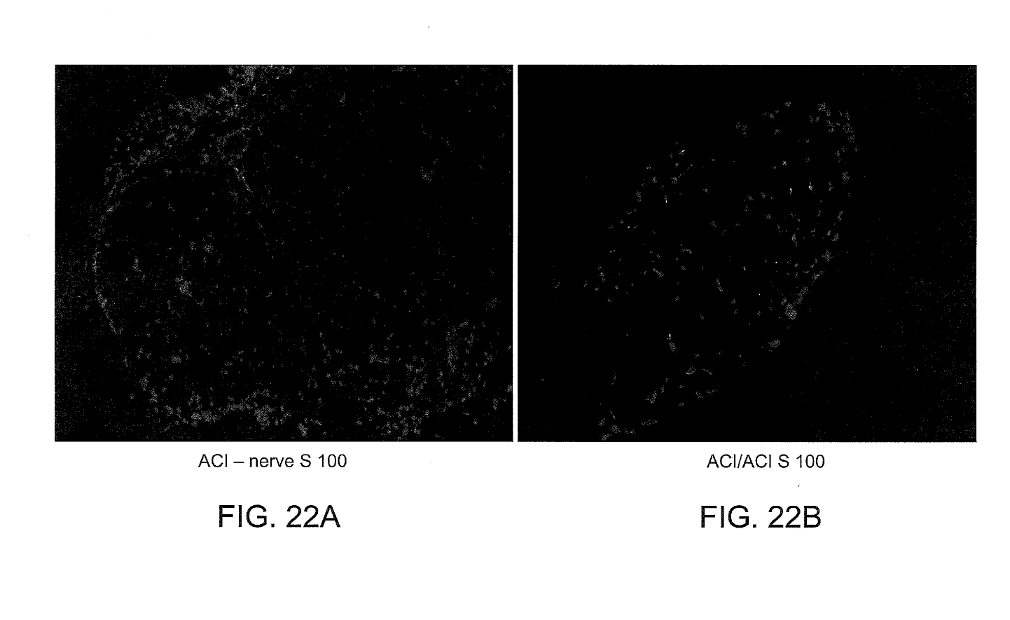

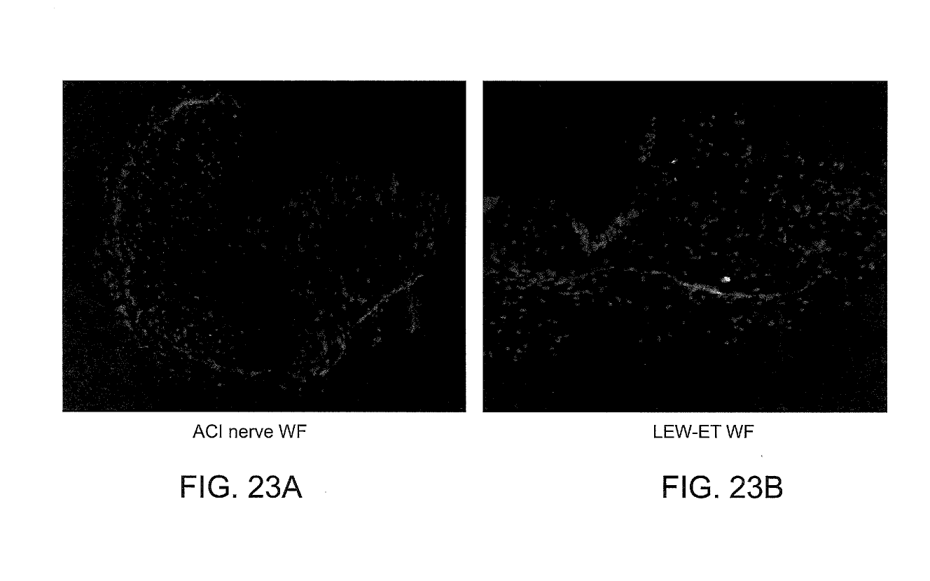

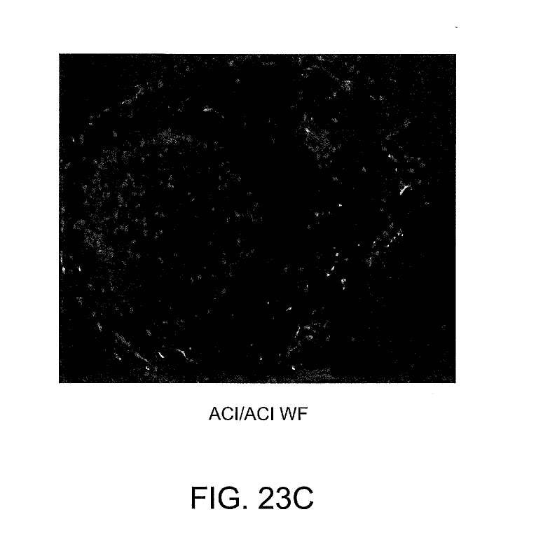

See FIGS. 16A-16D, 17A-17B, 18A-18C, 19A-19C, 20A-20C, 21A-21C, 22A-22B and 23A-23C.

### Epineurium

Epineurium from naïve LEW

| Antibody | Cross-section | Tube |
|---|---|---|
| NGF | Neg | neg |
| Laminin | + | ++ |
| S100 | w+ | w+ |
| GFAP | w+ | w+ |
| VEGF | + | + |
| vWF | + | + |
| MHC class I | w+ | w+ |
| MHC class II | Neg | neg |

w+—weak expression

### Example 3

### Synergistic Production of NGF And VEGF by Nerve Repair Biological Conduits—Study in vitro

### Objectives:

NGF and VEGF are important trophic factors in peripheral nerves regeneration. As described herein, bioconduits consisting of epineurium and bone marrow derived stromal cells (BMSC) improve peripheral nerve repair in rat sciatic nerve model. Increase of NGF and VEGF secretion was observed in site of transplantation of these conduits. Very little is known about to which extent natural microenvironment is vital for stimulation of these conduits to secrete NGF and VEGF. In the study presented here the secretion of NGF and VEGF by epineural tube alone and in the presence of BMSC was monitored in vitro for two weeks.

### Material and Methods:

Stromal cells were prepared from ACI rat bone marrow cells by seeding 30×10⁶ of bone marrow cells in 25 cm² flask using 10 ml alpha-MEM medium complete (containing 10% FBS). After first medium change (72 hrs) 2 cm sciatic nerve epineurium was added to flask with the stromal cells. Cultures containing stromal cells only and epineurium only were maintained in parallel. Media samples were collected every three days. Medium alone was used as a negative control. Levels of β-NGF and VEGF were determined with ELISA (R&D Systems).

### Results:

VEGF and NGF were detected in all culture media, e.g. in media from BMSC and epineuria. The secretion of both factors was increasing in all media. Interestingly, the levels of both factors in co culture were much higher than in media from BMSC and epineuria cultured alone.

### Conclusion:

The bioconduits consisting of epineurium and BMSC are able to expanded secretion of NGF and VEGF in vitro without additional stimulation from natural milieu of injured peripheral nerve. This feature can be applied to define of measure bioactivity of nerve repair conduits ex vivo.

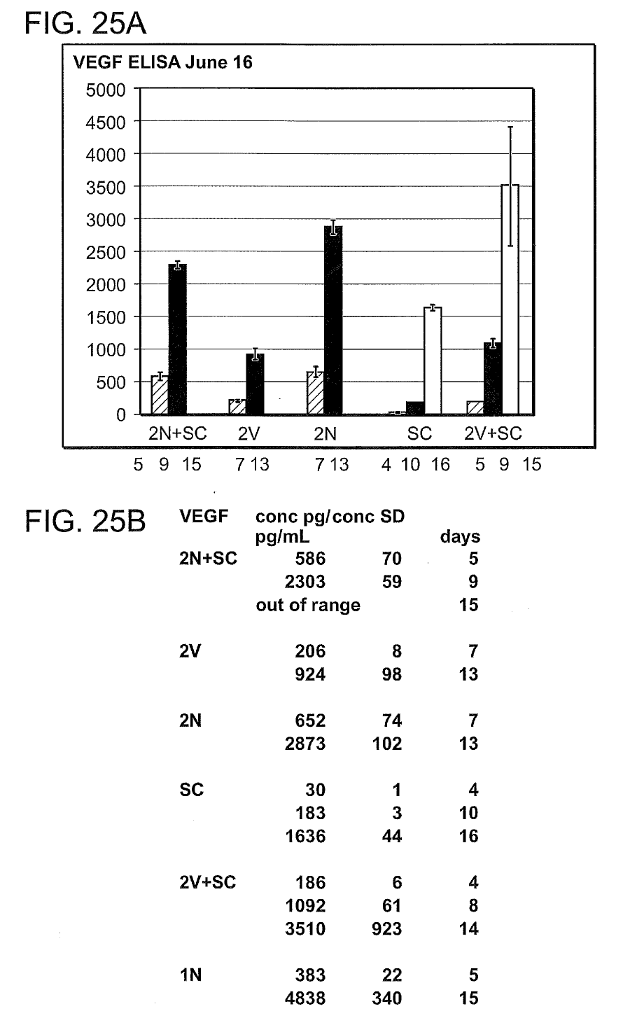

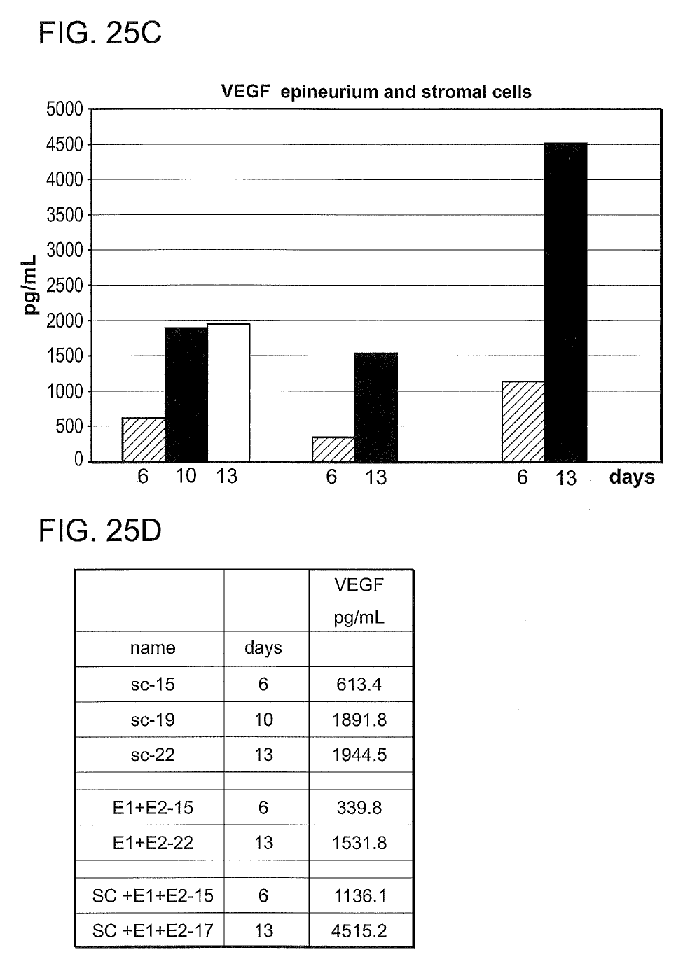

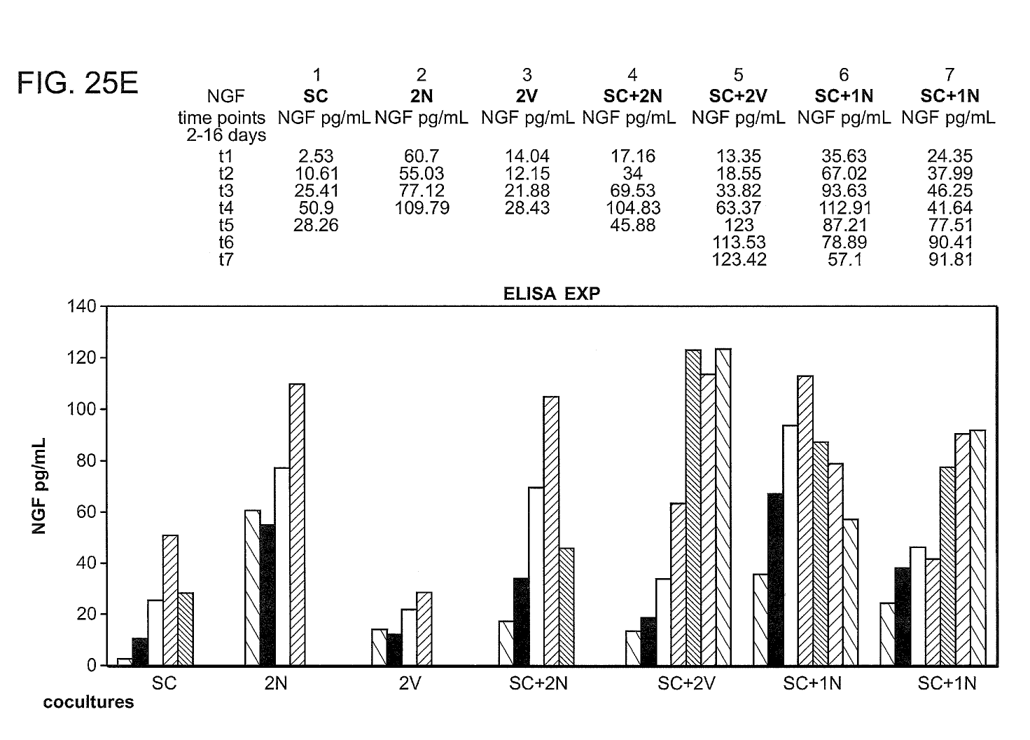

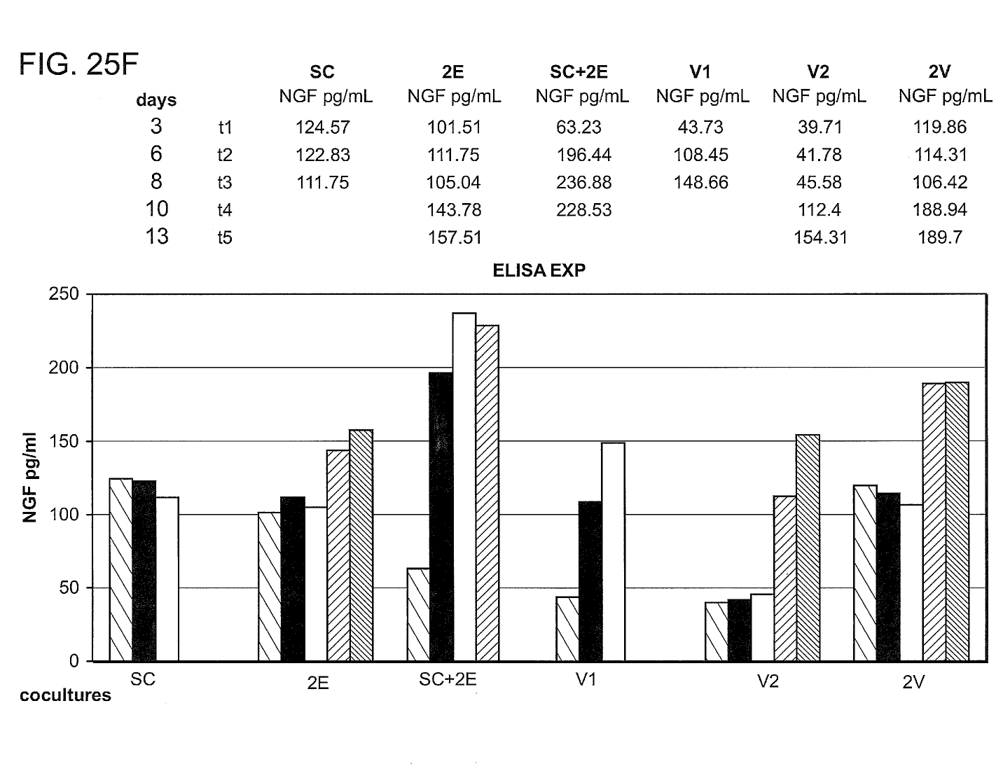

See FIGS. 25A-25F.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of generating a neural conduit comprising neurotrophic factors and angiogenic factors ex vivo comprising maintaining an isolated, naturally occurring epineural sheath under conditions in which neurotrophic factors and angiogenic factors are expressed in the epineural sheath, thereby generating a neural conduit comprising neurotrophic factors and angiogenic factors ex vivo.

2-3. (canceled)

4. The method of claim 1 comprising
a) introducing cells that enhance nerve regeneration into an isolated, naturally occurring epineural sheath, thereby producing a combination;
b) maintaining the combination under conditions in which neurotrophic factors and angiogenic factors are expressed in the epineural sheath,
thereby generating a neural conduit comprising neurotrophic factors and angiogenic factors ex vivo.

5. (canceled)

6. The method of claim 4 wherein the cells are stromal cells, chimeric cells or a combination thereof.

7. The method of claim 6 wherein the stromal cells are autologous stromal cells, isogenic stromal cells, allogenic stromal cells, xenogenic stromal cells or a combination thereof.

8. The method of claim 7 wherein the stromal cells are bone marrow cells, bone marrow stromal cells (BMSCs), mesenchymal stromal cells, mesenchymal fat cells or a combination thereof.

9. The method of claim 8 wherein the BMSCs are autologous BMSCs, isogenic BMSCs, allogenic BMSCs, xenogenic BMSCs or a combination thereof.

10-11. (canceled)

12. The method of claim 1 wherein the neural conduit is obtained from epineurium of a sensory nerve, a motor nerve or a combination thereof.

13. The method of claim 1 wherein the neural conduit is an autologous epineural tube, an allogenic epineural tube, an isogenic epineural tube, a xenogenic epineural tube or a combination thereof.

14-19. (canceled)

20. The method of claim 1 further comprising introducing one or more nerve growth factors, pro-angiogenic factors, cytokines or a combination thereof into the neural conduit.

21-25. (canceled)

26. The method of claim 4 further comprising transplanting the neural conduit in an individual in need thereof.

27. The method of claim 4 wherein the neurotrophic factors are CD31, LAM2, GFAP, S100, NGF or a combination thereof.

28. The method of claim 4 wherein the angiogenic factors are VEGF, vWF or a combination thereof.

29. A neural conduit produced by the method of claim 1.

30. A neural conduit produced by the method of claim 4.

31. A neural conduit comprising an isolated naturally occurring epineural sheath and saline, one or more epinueral strips, epineural powder or a combination thereof.

32. The neural conduit of claim 31 further comprising cells which enhance neural regeneration.

33. (canceled)

34. An article of manufacture comprising one or more isolated naturally occurring epineural sheaths, a device for introducing one or more agents which enhance neural regeneration into the one or more epineural sheaths, and instructions for use thereof.

35. The article of manufacture of claim 34 wherein the one or more agents is saline, one or more epineural strips, epinueral powder, cells or a combination thereof.

36. (canceled)

37. An article of manufacture comprising one or more neural conduits, wherein each neural conduit comprises an isolated naturally occurring epineural tube and one or more agents which enhance neural regeneration, and instructions for use thereof.

38. The article of manufacture of claim 37 wherein the one or more agents is saline, one or more epineural strips, epinueral powder, cells or a combination thereof.

39. The article of manufacture of claim 37 wherein the one or more neural conduits contains neurotrophic factors, angiogenic factors or a combination thereof, and instructions for use.
